(19) United States
(12) Patent Application Publication
(10) Pub. No.: US 2020/0170542 A1
(43) Pub. Date: Jun. 4, 2020

(54) SYSTEMS AND METHODS FOR POSITIONING AN INTRACRANIAL DEVICE USING BRAIN ACTIVITY

(71) Applicant: Ice Neurosystems, Inc., Washington, DC (US)

(72) Inventors: Allen Edward Waziri, Washington, DC (US); Ronald G. Emerson, New York, NY (US)

(73) Assignee: Ice Neurosystems, Inc., Washington, DC (US)

(21) Appl. No.: 16/631,760

(22) PCT Filed: Jul. 16, 2018
(86) PCT No.: PCT/US18/42298

§ 371 Date: Jan. 16, 2020

Related U.S. Application Data
(60) Provisional application No. 62/533,389, filed on Jul. 17, 2017.
(60) Provisional application No. 62/609,501, filed on Dec. 22, 2017.

(51) Int. Cl. A61B 5/06; A61B 5/00; A61B 5/0478; A61B 5/03

(52) U.S. Cl. CPC A61B 5/065; A61B 5/0042; A61B 5/0478; A61B 5/02; A61B 5/031; A61B 5/7435; A61B 2562/02; A61B 5/6868

(57) ABSTRACT

Systems and methods for positioning an intracranial device are disclosed. Certain embodiments of the invention encompass devices configured for implantation within the body that include elements responsible for detecting and transmitting electrical activity from the surrounding tissues and fluids. The system may include associated hardware and software designed for transmitting, processing, analyzing, and displaying relevant aspects of detected electrical activity. This information can be used throughout or following an insertion procedure to optimize or confirm device position within a particular intracranial location or tissue compartment.

### BACKGROUND OF THE INVENTION

### Field of the Invention

The invention encompasses systems and methods for placing and confirming position of an intracranial device through detection of brain activity. The described systems and methods provide real-time information to a physician and/or neurosurgeon during device placement in a patient and optimize subsequent function of a device and/or the capacity of a device to provide physiological monitoring within specific tissues of interest.

### Discussion of the Related Art

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Brain injury is often complicated by secondary physiological changes in the hours/days after the initial damage. Such secondary complications can include brain swelling, decreased blood flow, decreased oxygen, bleeding, infection, or seizures. These secondary complications, which may be preventable or reversible, are major sources of increased neurological morbidity and poor long-term neurological outcomes. Therefore, detecting these changes is an issue of central importance for treating patients with brain injuries.

Patients with acute neurological injuries often require implantation of devices into or around the brain that are designed to monitor and (in some cases) treat physiological alterations that influence brain health. In many cases these devices are external ventricular drains (EVD) that allow for measurement of intracranial pressure (ICP) and therapeutic drainage of cerebrospinal fluid (CSF). In other cases, small probes designed to measure cerebral oxygen concentration, temperature, blood flow, or important metabolites are placed at some depth within the brain tissue itself.

It is critical that such devices are placed within the appropriate intracranial tissue compartment. For example, a monitoring device that does not completely penetrate the brain tissue will provide spurious data. Alternatively, a device that is placed too far below the brain surface will not effectively monitor tissues of interest and may lead to increased risk of complication. Finally, an EVD that is not accurately placed within a cerebral ventricle, and more specifically that the entirety of the extent of the EVD encompassing the drainage holes is not within the cerebral ventricle, may not drain effectively and therefore provide limited benefit in reducing intracranial pressure.

Moreover, these devices are typically inserted into the cranium by a neurosurgeon under urgent or emergent conditions. Such procedures are most frequently performed at the bedside in the emergency room or intensive care unit, where dedicated surgical equipment and technical capacity are limited. Within these settings it is not possible to provide direct visualization of the intracranial tissues, so devices are therefore placed through small holes in the skull in “blind” fashion using basic external anatomic landmarks and a series of standardized techniques. Under these conditions, there are very limited real-time mechanisms for identifying or confirming device position within the brain tissue.

In some cases, EVD placement within a cerebral ventricle can be confirmed through visualization of CSF return from the catheter; however, spontaneous CSF flow may not occur even if the EVD is correctly positioned. In other cases, only a small portion of the drainage holes of the EVD may actually reside within the ventricle, allowing for some CSF flow but not allowing for optimal drainage or safe instillation of therapeutic compounds (e.g. tPA, antibiotics, etc) through the EVD. Gross distortion of intracranial structures (e.g., brain shift, presence of blood clots, etc) may also prevent accurate placement of devices. Subsequent brain “shift” due to swelling, enlargement of blood clots, or accumulation of fluid may also cause undetected displacement or movement of an implanted device in the hours to days following insertion.

The inability to accurately position intracranial monitoring devices can result in limited capacity to monitor the most metabolically active and functionally critical component of the brain, which is the gray matter of the cerebral cortex. The gray matter is only three to four millimeters thick in this region, making specific device targeting and localization to this anatomic compartment challenging (or impossible) even when direct visualization of the brain is permissible. In addition, the brain frequently “shifts” throughout normal and abnormal physiological processes (e.g. normal respiratory variation or brain swelling) and therefore there is frequently small concurrent shifts in the element of a device that is fixed in relation to the brain.

Given these considerations, confirmation of successful and accurate intracranial targeting for most devices placed at the bedside is dependent on post-procedural radiographic imaging. To obtain these images the insertion procedure must be completed, any surgical wounds closed and the patient transported to the appropriate location for brain imaging. In cases where a device is not appropriately positioned, as determined by radiographic imaging, a second procedure must be performed to reposition or replace a device. In still further cases, the area of intended implant may be too small to evaluate with standard imaging (e.g. the cerebral cortex using standard CT scanning) or otherwise poorly imaged due to anatomic constraints (e.g. cerebral cortex close to the skull which is often obscured by “bone artifact” on standard CT imaging).

Where repeat procedures must be performed to move or replace malpositioned devices, more than one “pass” through the brain must be undertaken. This significantly increases risk of secondary procedure-related brain injury as well as increasing delays in initiating device function that may carry significant risk for the patient. The need for a second or repeat procedure also leads to increased cost of supplies, excess time burden on physicians and nurses involved with patient care and potential delay of other life-saving interventions.

Additionally, in some cases the intent of a clinician is to place a device outside the brain tissue or dura mater (i.e. the epidural space), and in these cases it is deleterious for a device to be placed within the brain tissue or within/below/inside the dura mater.

Therefore, systems and methods that provide feedback regarding device position to the neurosurgeon throughout the insertion procedure, as well as during the subsequent period of device performance, are of critical value for maximizing function and safety while limiting secondary cost and complication.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Brain-derived oscillatory electrical activity is generated through the physiological activity of groups of neurons (the “generators”) located within specific anatomic locations within the head. A majority of these neurons are located in the gray matter of the cerebral cortex and are heavily interconnected through dense fiber bundles in the subcortical white matter. Patterns of electrical activity from these generators are often highly conserved in form and figure across individuals.

Specific anatomic compartments within the brain naturally and spontaneously generate oscillatory (and often patterned) electrical activity that can be detected, amplified, evaluated and displayed. The specific nature of brain-derived electrical signals recorded from the body can be dependent on a number of factors. Several of these factors include the “strength” of the generator (perhaps related to the number of neurons generating a specific signal), physical distance from the generator, characteristics of intervening tissue between the generator and the location of signal detection, “noise” from other generators or alternate sources of electrical activity, and physiological changes in the generator associated with external variables such as injury or medication. These factors can lead to stereotypical or predictable alterations in electrical signals recorded from a device positioned at particular geographic and/or anatomic locations related to a specific or more generalized generator.

Knowledge of consistent electrical activity patterns and inclusion of factors known to influence electrical activity in predictable fashion allows for consolidation of consistent, predictable, and/or reliable signatures of electrical activity to identify the position of an associated device within an intracranial tissue compartment. High fidelity data from systems designed to detect such electrical activity can be processed in automated and quantitative fashion using computer-based algorithms. Taken together, analysis of high fidelity electrical signals recorded from an intracranial device, combined with knowledge of predictable patterns of oscillatory activity that can be detected in discrete anatomic compartments of the brain, provides the system and method for confirming positioning of an intracranial device.

As a primary aspect of the invention, a device configured for implantation within the intracranial space would encompass elements along the physical structure of the implanted device designed to detect and transmit electrical signals generated by the brain, either in proximity to or at some distance from the implanted device. The implanted device may include at least one element designed to detect electrical activity, and may include a plurality of such elements.

In a preferred embodiment, the invention relates to a system for detecting the position of an implanted device within or around a compartment of a brain, wherein said system comprises an implanted device comprising a recording element capable of detecting and transmitting in real time brain activity, said implanted device connected by an interface to a processor capable of analyzing the position of the implanted device within or around the compartment of the brain.

Different compartments of the brain may be detected by the described system. Preferred brain compartments include: (a) gray matter; (b) white matter; (c) cerebral ventricle or other fluid containing space; (d) transition zone between gray matter and white matter; (e) transition zone between gray matter and a cerebral ventricle; (f) transition zone between white matter and a cerebral ventricle; (g) subdural or subarachnoid space; (h) epidural space; (i) local vasculature; (k) transition between bone, epidural space, subdural space, subarachnoid space, brain tissue, or fluid-containing space; (l) position within a specific geographic area of the brain in relationship to other structures or devices (including but not limited to anterior/posterior, medial/lateral, superior/inferior); (m) triangulated position of devices using data recorded from multiple sources; or (n) device proximity to or distance from any one of the compartments of (a)-(k).

In preferred embodiments, brain activity is measured by at least one of a parameter selected from (a) average voltage level; (b) root mean square (rms) voltage level and/or a peak voltage level; (c) derivatives involving fast Fourier transform (FFT) of recorded brain activity, possibly including spectrogram, spectral edge, peak values, phase spectrogram, power, or power ratio; also including variations of calculated power such as average power level, rms power level and/or a peak power level; (d) measures derived from spectral analysis such as power spectrum analysis; bispectrum analysis; density; coherence; signal correlation and convolution; (e) measures derived from signal modeling such as linear predictive modeling or autogressive modeling; (f) integrated amplitude; (g) peak envelope or amplitude peak envelope; (h) periodic evolution; (i) suppression ratio; (j) coherence and phase delays; (k) wavelet transform of recorded electrical signals, including spectrogram, spectral edge, peak values, phase spectrogram, power, or power ratio of measured brain activity; (l) wavelet atoms; (m) bispectrum, autocorrelation, cross bispectrum or cross correlation analysis; (n) data derived from a neural network, a recursive neural network or deep learning techniques; or (o) identification of the recording element(s) detecting local minimum or maximum of parameters derived from (a-n), for example as identified by waveform phase reversal in a bipolar chain of sequential adjacent sensors. In preferred embodiments, the brain activity is measured by categorical measurements, such as, for example, from volts (V), hertz (Hz), and/or or derivatives and/or ratios thereof.

In even further preferred embodiments, brain activity is measured by categorical measures of values, such as those selected from volts (V), hertz (Hz), and/or or derivatives and/or ratios thereof.

In further preferred embodiments, differences in the categorical measures indicate a change in brain activity of, for example at least 10%, of at least 20%, of at least 30%, of at least 40%, of at least 50%, of at least 90%, or at least 99% represents transition from: (a) gray matter to white matter; (b) gray matter to cerebral ventricle; (c) white matter to cerebral ventricle; (d) subdural/subarachnoid space to gray matter/white matter; (e) epidural space to subdural/subarachnoid space or gray matter/white matter; (f) cerebral vasculature in one compartment to cerebral vasculature in another compartment; or (g) or any combination of the above compartments.

In other preferred embodiments, the system can be updated in either a continuous or in a real-time fashion, and differences in the categorical measures at individual sensors, for example at least 10%, of at least 20%, of at least 30%, of at least 40%, of at least 50%, of at least 90%, or at least 99% represents movement of sensors within or between adjacent compartments.

In even further preferred embodiments, differences in the categorical measures indicate a change in brain activity of, for example at least 10%, of at least 20%, of at least 30%, of at least 40%, of at least 50%, of at least 90%, or at least 99% and represents the implanted device being positioned outside of the gray matter.

In further preferred embodiments, the implanted device further comprises a physiological sensor capable of measuring a physiological parameter. Examples of physiological parameter that can be measured and/or recorded include, but are not limited to intracranial pressure, oxygen concentration, glucose level, blood flow or tissue perfusion, tissue temperature, electrolyte concentration, tissue osmolarity, a parameter relevant to brain function and/or health, or any combination thereof.

It is expected that an implanted device comprising multiple recording elements will collect and/or record brain activity differently depending on numerous anatomical, positional, and/or functional parameters. Thus, it is expected recording elements on an implanted device may collect and record brain activity at different levels. Therefore, it is envisioned that the system will adjust and process brain activity recorded from “optimal” recording element(s) and/or will either disregard and/or minimize the processing of brain activity recorded from “sub-optimal” recording elements.

Thus, in a preferred embodiment, the system will measure, process, and/or display brain activity from an optimal recording element. In further preferred embodiments, the system will minimize and/or disregard brain activity measured from a sub-optimal recording element. This processing of brain activity from optimal recording elements can occur in real time and can also be dynamically adjusted to continuously identify those recording elements that are deemed optimal vs. sub-optimal to ensure recording of high quality brain activity.

Similarly, it is also expected that an implanted device comprising multiple alternative physiological sensors will collect and/or record physiological parameters differently depending on numerous anatomical, positional, and/or functional parameters. Thus, it is expected alternative physiological sensors on an implanted device may collect and record physiological parameters at different levels. Therefore, it is envisioned that the system will adjust and process physiological parameters recorded from “optimal” alternative physiological sensor(s) and/or will either disregard and/or minimize the processing of physiological parameters recorded from “sub-optimal” alternative physiological sensor(s).

Thus, in a preferred embodiment, the system will measure, process, and/or display physiological parameters from an optimal physiological sensor. In further preferred embodiments, the system will minimize and/or disregard physiological parameters measured from a sub-optimal physiological sensor. This processing of physiological parameters from optimal physiological sensors can occur in real time and can also be dynamically adjusted to continuously identify those physiological sensors deemed optimal vs. sub-optimal to ensure recording of high physiological parameters.

In preferred embodiments, the system updates in continuous or real-time fashion. Moreover, in further preferred embodiments, the system concurrently detects and processes: (a) brain activity in more than one brain compartment; or (b) brain activity and a physiological parameter in more than one brain compartment. Additionally, the processing of (a) brain activity or (b) brain activity and a physiological parameter can occur simultaneously.

Moreover, the implanted device can be designed for temporary, acute, semi-chronic, or chronic/permanent implantation in a patient. In other preferred embodiments, the implanted device can further have a therapeutic function. Preferred examples of such therapeutic function include, but are not limited to (a) the ability to drain or access a biological fluid, such as CSF, cyst fluid, or hematoma (i.e., a drainage function); (b) the ability to deliver a therapeutic agent; (c) the ability to deliver an electrical signal; and/or (d) any combination of the above.

In preferred embodiments, the physiological parameters are selected from: intracranial pressure, oxygen concentration, glucose level, blood flow or tissue perfusion, tissue temperature, electrolyte concentration, tissue osmolarity, a combination of the above, and/or an alternate method of monitoring designed to detect and display physiological parameters relevant to brain function and health. The implanted device can also be further capable of processing, filtering, amplifying, digitally transforming, comparing, displaying, storing, compressing, and/or providing a form of feedback regarding the monitored physiological parameter.

Thus, in further preferred embodiments, the implanted device comprises a drainage function, and preferably where recording elements are positioned proximal and/or distal to the drainage device. This implanted device can then be used in the system described herein to compare brain activity from the recording elements when inserted into, for example, a cerebral ventricle or other fluid space within the nervous system. In further preferred embodiments the system will identify congruence or dissimilarity in brain activity between these recording elements to confirm placement of the drainage function of the implanted device within a cerebral ventricle or other fluid containing space (e.g. cyst, cistern, hematoma cavity, etc.) within the nervous system.

The implanted device can be constructed from a number of different materials, including, but not limited to plastic, metal, organic, inorganic, and/or alternate compounds appropriate for implantation into the body. The implanted device can also be incorporated and/or is impregnated with a therapeutic substance, such as, for example, an antibiotic. The implanted device can also be flexible or rigid.

In preferred embodiments, the recording element is located: (a) in proximity with the tip of the implanted device; (b) in proximity with the structural portion of the implanted device designed to be positioned within the gray matter of the brain; (c) in proximity with the structural portion of the implanted device designed to be positioned within the white matter of the brain; (d) in proximity with the structural portion of the implanted device designed to be positioned within the subdural/subarachnoid space: (e) in proximity with the structural portion of the implanted device designed to be positioned within the epidural space: (f) in proximity with the structural portion of the implanted device designed to be positioned within a cerebral ventricle or other fluid containing space; (g) in proximity with the structural portion of the implanted device designed to be positioned within a blood vessel; (h) in proximity to the drainage function; and/or (i) any combination of the above.

In even further preferred embodiments, the implanted device comprises more than one recording element. In these situations, the multiple recording elements can be positioned: (a) dispersed along the implanted device; (b) located at the tip of the implanted device; (c) at least 50 um, 100 um, 200 um, 500 um, 750 um, 1 mm, 2 mm, 3 mm, 4 mm, 5 mm, 6 mm, 7 mm, 8 mm, 9 mm, 10 mm, 2 cm, 3 cm, 4 cm, 5 cm, 6 cm, 7 cm, 8 cm, 9 cm, 10 cm or any intervening distance apart from a another position sensor; (d) located in the white matter and a second position sensor is located in the gray matter; (e) located in the white matter, a second position sensor is located in the grey matter, and a third position sensor is located in a cerebral ventricle or other fluid space; and/or (f) physically separated from the implanted device and located elsewhere in/on the body or brain.

The implanted device can also further comprise a reference sensor capable of measuring a reference parameter, and in some embodiments, may include more than one reference sensor. In these situations, multiple reference sensors are (a) dispersed along the implanted device; (b) located at the tip of the implanted device; (c) at least 50 um, 100 um, 200 um, 500 um, 750 um, 1 mm, 2 mm, 3 mm, 4 mm, 5 mm, 6 mm, 7 mm, 8 mm, 9 mm, 10 mm, 2 cm, 3 cm, 4 cm, 5 cm, 6 cm, 7 cm, 8 cm, 9 cm, 10 cm or any intervening distance apart from a second sensor; (d) located in the white matter and a second reference sensor is located in the gray matter; (e) located in a cerebral ventricle or other fluid containing space; (f) physically separated from the implanted device and located elsewhere in/on the body or brain; and/or (g) located proximal and/or distal to the drainage function.

In further preferred embodiments, the implanted device can comprise more than one physiological sensor. In these situations, multiple physiological sensors are: (a) dispersed along the implanted device; (b) located at the tip of the implanted device; (c) at least 50 um, 100 um, 200 um, 500 um, 750 um, 1 mm, 2 mm, 3 mm, 4 mm, 5 mm, 6 mm, 7 mm, 8 mm, 9 mm, 10 mm, 2 cm, 3 cm, 4 cm, 5 cm, 6 cm, 7 cm, 8 cm, 9 cm, 10 cm or any intervening distance apart from a second sensor; (d) located in the white matter and a second sensor is located in the gray matter; (e) located in a cerebral ventricle or other fluid containing space; and/or (f) physically separated from the implanted device and located elsewhere in/on the body or brain.

In preferred embodiments, the implanted device is placed through the skin, bone, dura, brain tissue, fluid spaces, cerebral blood vessels or other body tissues.

In further preferred embodiments, the processor is capable of processing, filtering, amplifying, digitally transforming, comparing, storing, compressing, displaying, and/or otherwise transmitting (a) the brain activity; (b) the brain activity and the physiological parameter; (c) the brain activity, the physiological parameter, and the reference parameter; or (d) the brain activity and the reference parameter.

In preferred embodiments, the implanted device, the interface and the processor are integrated with one another. In other embodiments, the processor and the interface are integrated with one another. And in still other preferred embodiments, the implanted device and the interface are integrated with one another.

As described herein, the interface connects the implanted device to the processor. The interface connection may be either a physical connection or a wireless connection. In certain preferred embodiments, the interface may be implanted in the patient. In further preferred embodiments, the interface is capable of processing, filtering, amplifying, digitally transforming, compressing and/or transmitting (a) the brain activity; (b) the brain activity and the physiological parameter; (c) the brain activity, the physiological parameter, and the reference parameter; or (d) the brain activity and the reference parameter.

In further preferred embodiments, the system further comprises an independent power source. In other preferred embodiments, the processor further comprises hardware and/or software that analyzes, manipulates, displays, correlates, stores and/or otherwise transmits (a) the brain activity; (b) the brain activity and the physiological parameter; (c) the brain activity, the physiological parameter, and the reference parameter; or (d) the brain activity and the reference parameter. The hardware can also further comprise a power supply, a central processing unit/motherboard, memory components, data/media storage capacity, video/graphics card, a sound card, input and output peripherals, physical connections for wired transmission and/or a wireless interface.

In other preferred embodiments, the system can further comprise at least one alternate physiological monitoring device. Such alternate physiological monitoring device can also be connected to the system via an interface, such as via a physical or a wireless interface. Examples of such physiological monitoring devices include, but are not limited to, a heart rate monitor, a EKG measurement device, an oximeter, combined heart rate and oximeter device such as a pulse oximeter, a body temperature sensor, a blood pressure measurement device, a neuronal activity measurement device, an EEG measurement device, or other physiological recording systems and combinations thereof.

In further specific embodiments, the processor is capable of inputting, recording, integrating, analyzing, compressing, storing, displaying, transmitting and/or utilizing data selected from: (a) alternate physiological monitoring device implanted within or around the brain, including, but not limited to an oxygen sensor, a blood perfusion sensor, a brain metabolites sensor, a temperature sensor, or an intracranial pressure sensor; (b) a system designed for monitoring aspects of physiology not directly recorded from the brain, including, but not limited to: heart rate monitor; EKG measurement device; temperature sensor; combined heart rate and oximeter device such as a pulse oximeter; blood pressure measurement device; or other physiological recording system and combinations thereof; (c) a source of electroencephalography or electrocorticography such as that recorded from standard scalp or subdural electrodes; (d) associated clinical interventions such as medications, ventilator settings, or temperature management; and/or (e) a patient's medical record.

In further preferred embodiments, the system further comprises a display component. The display component can be capable of displaying: (a) at least one native or processed brain activity detected by the implanted device; (b) at least one aspect of brain physiology detected by a concurrent physiological monitor associated with the implanted device; (c) at least one aspect of brain physiology detected by a physiological monitor directly associated with the brain not directly associated with the system; (d) at least one aspect of other physiological data recorded from the patient not directly associated with the brain, such as heart rate, systemic oxygen saturation, blood pressure or other vital signs; (e) at least one aspect of other clinical information associated with the patient, such as demographic data or medications being administered; (f) data associated with the function of the system as a whole, such as specific recording elements in use, location of selected recording elements within particular compartments of the brain, specifics regarding analytics of displayed brain activity, system power level and/or related variables; (g) the brain activity; (h) the brain activity and the physiological parameter; (i) the brain activity, the physiological parameter, and the reference parameter; and/or (j) the brain activity and the reference parameter.

In other preferred embodiments, the system can provide auditory or visual information. Such auditory or visual information provides information, such as, for example: (a) the position of the implanted device or an aspect of the implanted device within a compartment of the brain; (b) the settings or function of the system; (c) changes associated with monitored brain activity or associated physiological variables; (d) factors controlled by the user with regard to the function or display capabilities of the system; (e) visual information regarding implanted device position; (f) auditory feedback regarding implanted device position; (g) feedback enabling alteration of settings or performance of the system; (h) the brain activity; (i) the brain activity and the physiological parameter; (j) the brain activity, the physiological parameter, and the reference parameter; and/or (k) the brain activity and the reference parameter.

In other preferred embodiments, the system can be additionally configured for wireless transmission of data to a local server or cloud-based system. Examples of such data, include, but are not limited to: (a) unprocessed or processed brain activity; (b) other physiological monitors; (c) documentation of associated clinical interventions; (d) other patient-specific factors; (e) the brain activity; (f) the brain activity and the physiological parameter; (g) the brain activity, the physiological parameter, and the reference parameter; and/or (h) the brain activity and the reference parameter.

The system may also comprise a graphical user interface (GUI), which in some instances, would allow the user to modify variables associated with the system. Examples of such variables include, but are not limited to: (a) aspects of real-time feedback regarding the position of the implanted device; (b) the ability to allow the user to select or modify elements of the display function; (c) the ability to allow the user to select or modify elements of the recording or reference function; (d) the ability to allow the user to select or modify elements of the system processor with regard to aspects of recorded brain activity analysis; (e) the ability to allow the user to enter additional data or patient information; (f) the ability to allow the user to select or modify alarms or indicators; and/or (g) the ability to allow the user to otherwise modify the input, output, storage, analytical, display or recording function of the system.

The system can also comprise software, such as, for example: (a) software designed to detect and display specific electrical patterns or signals of measured brain activity; (b) software designed to calculate and display integrated amplitude of recorded electrical signals of measured brain activity; (c) software designed to calculate and display peak envelope or amplitude peak envelope of recorded electrical signals of measured brain activity; (d) software designed to calculate and display periodic evolution within recorded electrical signals of measured brain activity; (e) software designed to calculate and display suppression ratios within recorded electrical signals of measured brain activity; (f) software designed to calculate and display coherence and phase delays (g) software designed to calculate and display fast Fourier transform of recorded electrical signals, such as for example, FFT; possibly including spectrogram, spectral edge, peak values, phase spectrogram, power, or power ratio of measured brain activity; (h) software designed to calculate and display wavelet transform of recorded electrical signals, possibly including spectrogram, spectral edge, peak values, phase spectrogram, power, or power ratio of measured brain activity; (i) software designed to calculate and display wavelet atoms associated with recorded electrical signals of measured brain activity; (j) software designed to calculate and display bispectrum, autocorrelation, cross bispectrum or cross correlation analysis of recorded electrical signals of measured brain activity; (k) software designed to calculate and display signals from isolated frequency bands of oscillatory electrical activity of measured brain activity; (l) software designed to calculate and display ratios comparing elements of variation in particular frequency bands of oscillatory electrical activity of measured brain activity; (m) software designed to calculate and display relative levels of activity in individual frequency bands of oscillatory electrical activity of measured brain activity; (n) software that utilizes neural networks, recursive neural networks or deep learning techniques; (o) software to identify a sensor recording local minimum or maximum of parameters derived from (a-n), for example as identified by waveform phase reversal in a bipolar chain of sequential adjacent sensors; (p) software designed to record and/or measure the brain activity; (q) software designed to record and/or measure the brain activity and the physiological parameter; (r) software designed to record and/or measure the brain activity, the physiological parameter, and the reference parameter; (s) software designed to record and/or measure the brain activity and the reference parameter; and/or (t) software to measure changes in real time any one of the parameters derived from (a)-(s).

### BRIEF DESCRIPTION OF THE FIGURES

The objects and features of the invention can be better understood with reference to the following detailed description and accompanying drawings.

The objects and features of the invention can be better understood with reference to the following detailed description and accompanying drawings.

FIG. 1 is a schematic view of an implanted device positioned within various compartments of the brain.

FIG. 2 is a schematic view of the implanted device connected to an interface and processor.

FIG. 3 is a schematic view of an implanted device including a physiological device capable of monitoring a physiological parameter.

FIG. 4 is a schematic view showing an alternative arrangement of an implanted device including a physiological device capable of monitoring a physiological parameter.

FIG. 5 is a schematic view of the system showing the implanted device, the interface and the processor along with an alternate physiological device capable of monitoring physiological parameters.

FIG. 6 is a schematic view of an alternative arrangement of the system showing the implanted device, the interface, and the processor, wherein the interface and the processor are wirelessly connected.

FIG. 7 is a schematic view of an alternative arrangement of the system showing the implanted device and interface implanted under the skin of a patient and connected wirelessly to the processor.

FIG. 8 is a flow chart outlining one exemplified embodiment of the interface between the implanted device and the processor, including connection inputs, amplifiers, filters, converters, processors, interfaces and outputs.

FIG. 9 is a flow chart outlining one exemplified embodiment of the processor unit including inputs, various connected devices, user interface, display and outputs.

FIG. 10 is a schematic view of an implanted device comprising a drainage function, where recording elements are positioned proximal and distal to the drainage function.

FIG. 11 illustrates representative raw EEG data recorded from an electrode array spanning a cerebral cortex using a common extracranial recording reference. Contacts are located within white matter (WM), gray matter (GM), subdural space (SD) and epidural space (ED).

FIG. 12 provides representative data uV/Hz and square root of uV/Hz from an electrode array spanning a cerebral cortex using a common extracranial reference demonstrating comparative numerical differences between contacts in white matter (WM), gray matter (GM), subdural space (SD) and epidural space (ED).

FIG. 13 provides a compressed spectral array generated by Fast Fourier Transform of data recorded from an electrode array spanning a cerebral cortex using a common extracranial reference, demonstrating visual differences in EEG power (red highest power, blue lowest power) between white matter (WM), gray matter (GM), subdural space (SD) and epidural space (ED).

FIG. 14 illustrates raw EEG data recorded from an electrode array spanning a cerebral cortex using a bipolar (adjacent contact) referencing strategy. Recorded channels represent pairs of electrodes located in white matter/white matter (WM/WM), white matter/gray matter (WM/GM), gray matter to gray matter (GM/GM), gray matter to subdural space (GM/SD) and subdural space to epidural space (SD/ED).

FIG. 15 provides representative uV/Hz and square root of uV/Hz from an electrode array spanning a cerebral cortex using a bipolar (adjacent contact) referencing strategy. Recorded channels represent pairs of electrodes located in white matter/white matter (WM/WM), white matter/gray matter (WM/GM), gray matter to gray matter (GM/GM), gray matter to subdural space (GM/SD) and subdural space to epidural space (SD/ED).

FIG. 16 provides a compressed spectral array generated by Fast Fourier Transform of data recorded from an electrode array spanning a cerebral cortex using a bipolar referencing strategy (adjacent contact). Recorded channels represent pairs of electrodes located in white matter/white matter (WM/WM), white matter/gray matter (WM/GM), gray matter to gray matter (GM/GM), gray matter to subdural space (GM/SD) and subdural space to epidural space (SD/ED).

FIG. 17 illustrates raw EEG data recorded from an electrode array spanning gray matter of cerebral cortex (GM), subcortical white matter (WM) and periventricular gray matter (PVGM) using a bipolar referencing strategy (adjacent contact).

FIG. 18 represents calculated total power over time of EEG recorded from an electrode array spanning a cerebral cortex using a bipolar referencing strategy (adjacent contact), demonstrating relative power between paired contacts in gray matter/gray matter (GM/GM), white matter/gray matter (WM/GM), gray matter/subdural space (GM/SD), white matter/white matter (WM/WM), subdural space to epidural space (SD/ED) and epidural space/epidural space (ED/ED).

FIG. 19 provides representative power values at a single time point within discrete frequency bands recorded from electrodes within separate intracranial compartments including white matter, white/gray junction and gray matter using a bipolar referencing strategy (adjacent contact). Delta band includes 1-4 Hz, theta band includes 4-8 Hz, alpha band includes 8-13 Hz, beta/gamma band includes 13-30 Hz. Power values are all noted at a factor of 10⁷.

FIG. 20 provides comparative analysis of calculated power ratios from EEG recorded from electrodes using a bipolar referencing strategy (adjacent contacts) located within subcortical white matter as well as cortical gray matter.

### DETAILED DESCRIPTION

### Definitions

The following definitions are provided for specific terms which are used in the following written description.

As used in the specification and claims, the singular form “a”, “an” and “the” include plural references unless the context clearly dictates otherwise. For example, the term “an element ” includes a plurality of elements.

As used herein, the term “comprising” is intended to mean that the system, implanted device, processor, and/or interference and/or methods described herein include the recited elements, and can include other elements. “Consisting essentially of”, when used to define the system, implanted device, processor, and/or interference and/or methods described herein, shall mean excluding other elements of essential significance to the combination. “Consisting of” shall mean excluding more than elements and substantial method steps for use of the system. Embodiments defined by each of these transition terms are within the scope of this invention.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to systems or processes, the term can mean within an order of magnitude, preferably within 5 fold, and more preferably within 2 fold, of a value. Unless otherwise stated, the term ‘about’ means within an acceptable error range for the particular value, such as ±1-20%, preferably ±1-10% and more preferably ±1-5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

As used herein, a “subject” is a vertebrate, preferably a mammal, more preferably a human. Mammals include, but are not limited to, murines, simians, humans, farm animals, sport animals, and pets. In other preferred embodiments, the “subject” is a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), or an ape (e.g., gorilla, chimpanzee, orangutan, gibbon). In other embodiments, non-human mammals, especially mammals that are conventionally used as models for demonstrating therapeutic efficacy in humans (e.g., murine, primate, porcine, canine, or rabbit animals) may be employed.

As used herein, a “compartment” or a “compartment of a brain” or a “brain compartment” is defined both anatomically and spatially. For example, anatomic brain compartments that can be measured by the system described herein include, but are not limited to: (a) gray matter; (b) white matter; (c) cerebral ventricle or other fluid containing space; (d) transition zone between gray matter and white matter; (e) transition zone between gray matter and a cerebral ventricle; (f) transition zone between white matter and a cerebral ventricle; (g) subdural or subarachnoid space; (h) epidural space; (i) local vasculature; (k) transition between bone, epidural space, subdural space, subarachnoid space, brain tissue, or fluid-containing space; (l) position within a specific geographic area of the brain in relationship to other structures or devices (including but not limited to anterior/posterior, medial/lateral, superior/inferior); (m) triangulated position of devices using data recorded from multiple sources; or (n) device proximity to or distance from any one of the compartments of (a)-(k).

However, the skilled artisan also recognizes that anatomic compartments positioned in different locations in the brain are not always equivalent. For example, grey matter located in the cerebral cortex is not identical to grey matter located in the thalamus. The system described herein as described herein is able to recognize and/or identify the different compartments of the brain, both anatomically and spatially.

As used herein, the “implanted device” is designed for insertion into the human body by a surgeon or other clinician with the intent of delivering and/or providing treatment, monitoring brain activity and/or other physiological functions, and/or combinations thereof. The implanted device comprises a recording element(s) and/or may include other elements designed and/or configured to detect and transmit electrical signals reflecting brain activity. These elements may be constructed of metal, plastic, or other compounds.

As used herein, a “recording element” is a contact which is capable of detecting brain electrical activity. Preferably, the recording element is metallic.

As used herein, a “reference element” is a contact (preferably also made of metal) designed to act as a control allowing for the comparison of brain activity detected by one or more recording elements on the implanted device.

As used herein, a “processor” is capable of modifying, analyzing, correlating, storing and displaying recorded brain electrical activity to identify, in real time, the position of the implanted device within or around a compartment of the brain. The processor may comprise hardware and/or software elements.

As used herein, a “drainage function” refers to a structure on the implanted device that allows for the removal of and/or access to a biological fluid, such as CSF, cyst fluid or hematoma.

As used herein, “brain activity” is defined as the electrical signals generated by the brain. As described herein, “brain activity” or “brain electrical activity” can be measured by a variety of different parameters capable of detecting and/or measuring electrical activity, including, but not limited to: (a) average voltage level; (b) root mean square (rms) voltage level and/or a peak voltage level; (c) derivatives involving fast Fourier transform (FFT) of recorded brain activity, possibly including spectrogram, spectral edge, peak values, phase spectrogram, power, or power ratio; also including variations of calculated power such as average power level, rms power level and/or a peak power level; (d) measures derived from spectral analysis such as power spectrum analysis; bispectrum analysis; density; coherence; signal correlation and convolution; (e) measures derived from signal modeling such as linear predictive modeling or autogressive modeling; (f) integrated amplitude; (g) peak envelope or amplitude peak envelope; (h) periodic evolution; (i) suppression ratio; (j) coherence of calculated values such as spectrogram, spectral edge, peak values, phase spectrogram, power, and/or power ratio; (k) wavelet transform of recorded electrical signals, including spectrogram, spectral edge, peak values, phase spectrogram, power, or power ratio of measured brain activity; (l) wavelet atoms; (m) bispectrum, autocorrelation, cross bispectrum or cross correlation analysis; or (n) waveform phase reversal, or other alteration of waveform characteristics related to dipole, resulting in variable positive or negative values between recording elements and reference sensors at specific moments in time. In preferred embodiments, the brain activity is measured by categorical measurements, such as, for example, from volts (V), hertz (Hz), and/or or derivatives and/or ratios thereof.

As used herein, the system can provide information regarding brain activity in a “continuous” and/or in a “real-time” fashion, allowing for optimized detection of brain activity and/or positioning of the implanted device in a brain compartment.

As used herein, the implanted device is designed for temporary (i.e., minutes to hours), acute (i.e., hours to days), semi-chronic (i.e., days to weeks), or chronic/permanent (i.e., weeks and beyond) implantation in a patient.

As used herein the recording element may be positioned “in proximity with” other elements on the implanted device. “In proximity with” is defined as “at, within or associated with” the specified element.

For example, as described herein, the implanted device may further comprise a reference sensor which allows for comparison of brain activity detected by multiple recording elements.

As described herein, a “physical interface” includes, but is not limited to, elements such as connectors, filters, amplifiers, analog-to-digital converters, or other hardware and software elements capable of transmitting brain activity detected by a recording element(s) on the implanted device to the processor.

As used herein, a “wireless interface” may also include elements such as connectors, filters, amplifiers, analog-to-digital converters, or other hardware and software elements capable of transmitting brain activity detected by a recording element(s) on the implanted device to the processor. As used herein, the term “wireless” or “wireless pathway” shall refer to an energy and/or information transmission pathway that does not include or otherwise rely on a physical conduit for transmission, such as an electromagnetic, sound and/or light transmission of energy and/or information that passes through the tissue of a patient without the use of a physical conduit.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the system in use and/or operation in addition to the orientation depicted in the figures. For example, if the system in a figure is turned over, elements described as “below” and/or “beneath” other elements or features would then be oriented “above” the other elements or features. The system can be otherwise oriented (e.g., rotated 90 degrees or at other orientations) and the spatially relative descriptors used herein interpreted accordingly.

### Detailed Description

An embodiment of the invention includes a system and method allowing for confirmation of intracranial device positioning through recording and analysis of electrical signals generated by the brain.

Systems and methods associated with the invention may be designed to detect, analyze and display elements of spontaneous electrical activity of the brain to guide and confirm device position within the intracranial space. While the invention will be described for use with devices placed at the bedside in patients with acute neurological injuries, the invention may be applicable for device placement in other settings such as ventricular shunt placement, CSF reservoir placement, intraparenchymal catheter placement as performed for convection enhanced delivery of compounds, spinal drain/catheter insertion, epidural catheter placement in the head or spine, channels designed to optimize neurosurgical operative procedures, intra-/endovascular catheters or associated devices and stents, subcutaneous electrodes or recording devices,

As an example of a particular embodiment of the invention, an implanted device (such as an EVD) encompassing a recording element(s) at the distal end (the portion intended for intraventricular positioning) or encompassing recording elements at the distal and proximal ends of the portion intended for intraventricular positioning is attached via a wired interface comprising elements capable of converting, processing, and transmitting the detected electrical activity in real time to the processor. In one example, the data can then be converted into a (visual) signal with/without associated additional (auditory) cues on a display component of the processor, which indicates the position of the tip or the entirety of the implanted device within a particular anatomic compartment.

As described herein and in preferred embodiments, the neurosurgeon or clinician would progressively advance the tip of the implanted device until a signal confirming that the desired intraventricular position of the distal end of the implanted device or the entirety of the drainage function of the device has been reached. At that point, the implanted device could be secured in place for subsequent use in monitoring and drainage. During device insertion, real-time analysis of recorded electrical signals would provide feedback (visual and/or auditory) to the neurosurgeon confirming that the implanted device was appropriately placed within the brain tissue (rather than within the epidural or subdural space). Further analysis could also provide information regarding position within the white or gray matter.

In another embodiment of the invention the implanted device would be attached to the processor via an interface designed to locally process and transmit detected electrical activity from the implanted device. Components of this interface could either be external to the patient or implantable under the patient's skin. Information could then be transmitted wirelessly to the processor for further processing, display and utility, as per the prior example.

Further, in preferred embodiments, initial processing of the electrical signals prior can occur in the interface, rather than in the processor. Examples of such “initial processing” include, but are not limited to signal amplification, bandpass or other filtering, analog to digital conversion, etc. Thus, the interface may also be configured to provide some basic processing of the electrical signal and can also provide some auditory or visual feedback to the neurosurgeon.

In another preferred embodiment of the invention, intracranial pressure can be measured by a recording element(s) on the implanted device.

Following insertion of the implanted device, continuous monitoring would confirm continued appropriate positioning of the implanted device within the brain tissue. Notably, any shifting of the brain or movement of the implanted device may result in egress of the device from the brain tissue and/or cerebral ventricle resulting in spurious data or ineffective CSF drainage, respectively. Continuous analysis of electrical signals detected by the implanted device would provide notification that the implanted device is sub-optimally positioned.

In another embodiment of the invention, the recording element is capable of monitoring a physiological variable relevant to neuronal health (such as, for example oxygen or glucose) or a relevant physiological parameter that can be detected within the CSF. Real-time analysis of detected electrical information could then be used to identify/confirm position of the implanted device within the gray matter of the cerebral cortex (rather than, for example, subdural space or white matter). Continuous recording and monitoring during the ongoing period of the implanted device would allow for confirmation of the appropriate position within the desired brain compartment.

Another iteration would use contacts on the tip of a catheter placed endovascularly—detected electrical signals would be used to guide or position a catheter or other device within the cerebral blood vessels to an appropriate location.

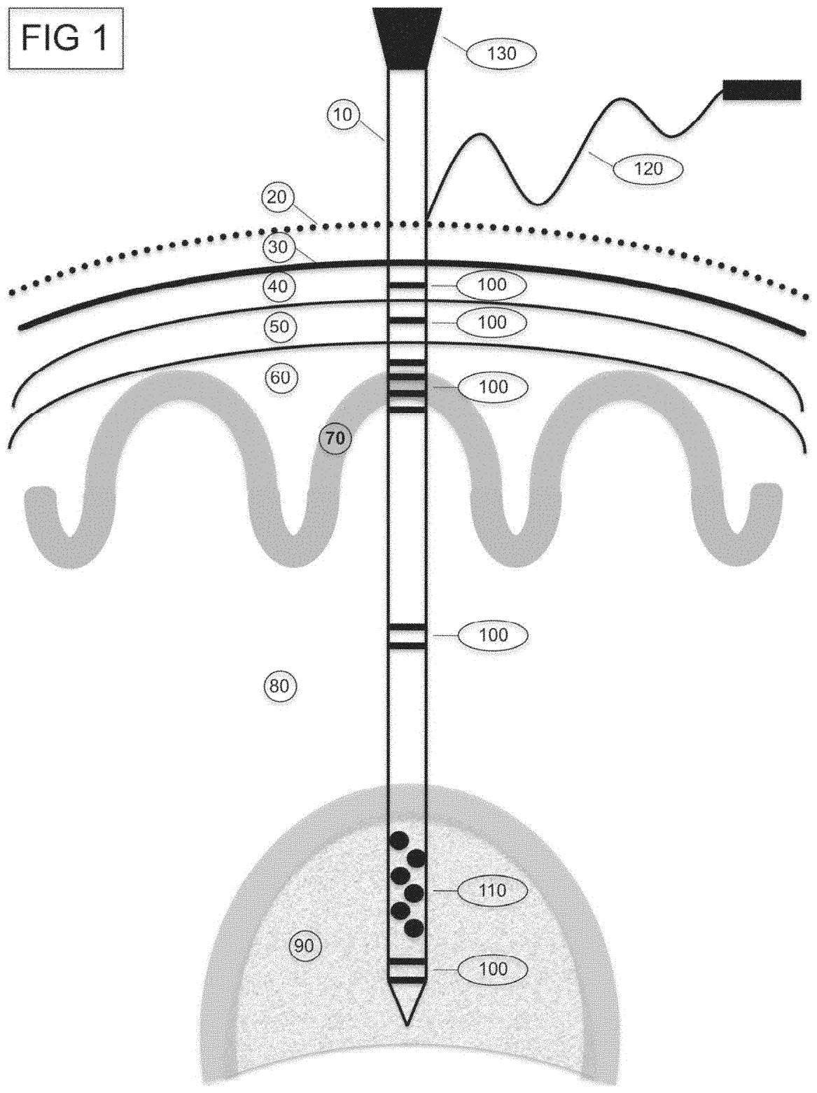

Referring now to FIG. 1, an implanted device (10) is shown placed through the skin (20), the bone of the skull (30), the Epidural space (40), the Subdural space (50); the Subarachnoid space (60), the Gray Matter of Cerebral Cortex (70), the White Matter (80) and into the Cerebral ventricle (90). In this embodiment, the recording elements (100) are positioned along the shaft of the implanted device (10) to measure brain activity in the Epidural space (40), the Subdural space (50), the Subarachnoid space (60), the Gray Matter of Cerebral Cortex (70), the White Matter (80), and the Cerebral Ventricle (90). FIG. 1 also shows one embodiment where the implanted device (10) transmits the brain activity recorded by the recording elements (100) via a wire (120).

FIG. 1 also shows a preferred embodiment, wherein the implanted device (10) also has a therapeutic function. In this example, the therapeutic function allows for the drainage of cerebrospinal fluid (CSF), providing relief of elevated intracranial pressure. Here, the implanted device (10) also comprises drainage holes (110) preferably positioned at the tip of the implanted device (10). The implanted device (10) can then serve the dual function of draining CSF along with recording brain activity. In this embodiment, the implanted device (10) will also comprise a connection point (130) for a catheter to drain the CSF.

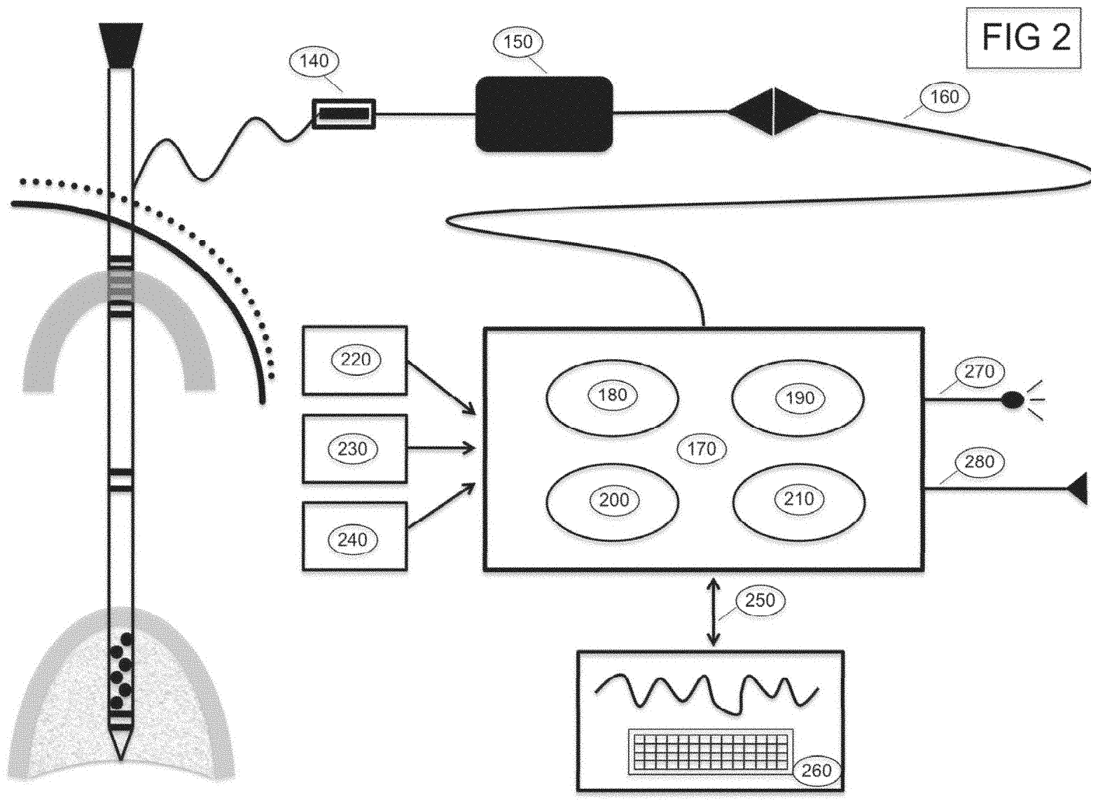

FIG. 2 shows the implanted device of FIG. 1 connected to the interface (150) and a hardware element containing the processor (170). As described herein, the Interface (150) can comprise, for example, an amplifier, a filter, and/or an analog-to-digital converter. Additionally, in this preferred embodiment, an adaptor (140) connects wires from the recording elements (100) on the implanted device (10) to the interface (150). FIG. 2 also shows further preferred embodiment whereby the interface (150) is connected to the hardware unit containing the processor (170) by a wired connection (160). The system shown in FIG. 2 illustrates further embodiments whereby a computer hardware system (170) comprises the processor (180), a data storage element (190), a means for interacting with a display element (200) (such as, for example, a sound and/or video card), and a means for inputting and/or outputting data (210) (e.g., input/output peripherals).

The system shown in FIG. 2 also illustrates a preferred embodiment whereby the system further comprises at least one alternate physiological monitoring device (220) capable of monitoring a physiological parameter. An example of such alternate physiological monitoring device (220) includes, but is not limited to a blood pressure or heart rate sensor.

The system shown in FIG. 2 also illustrates further preferred embodiments whereby the system comprises a means for connecting to an external EEG system (230), to a hospital's electronic record system (240), and/or to a display, auditory output and/or interactive user element (260). The connections (250) between these components (220, 230, 240, 260) can be wired or wireless as described herein.

FIG. 2 also illustrates that the system, in preferred embodiments, can be capable of wireless transmission of data to external server or cloud-based system (270) and/or wired transmission of data to local server or network (280).

In FIG. 2, exemplifies that recorded brain activity can be amplified, filtered and undergo analog-to-digital conversion via the interface (150) and the resulting signal can then be transmitted through a wired connection (160) to a hardware element (170) containing the processor (180) and optionally associated additional features of the system (190, 200, 210). Additional data can also be inputted into the system; such data, includes, but is not limited to alternate physiological monitoring device (220), electroencephalography (230), or the hospital electronic medical system (240). This data can be processed and sent in various forms to a display component (260) for observation and interpretation by a clinician user. The display element (260) can also include a user interface capable of allowing the clinician to alter the display functions or other aspects of system function. Data can be stored internally (such as, for example 190) or sent via wired (280) or wireless transmission (270) to external devices, a local server, a local network or a cloud-based data system.

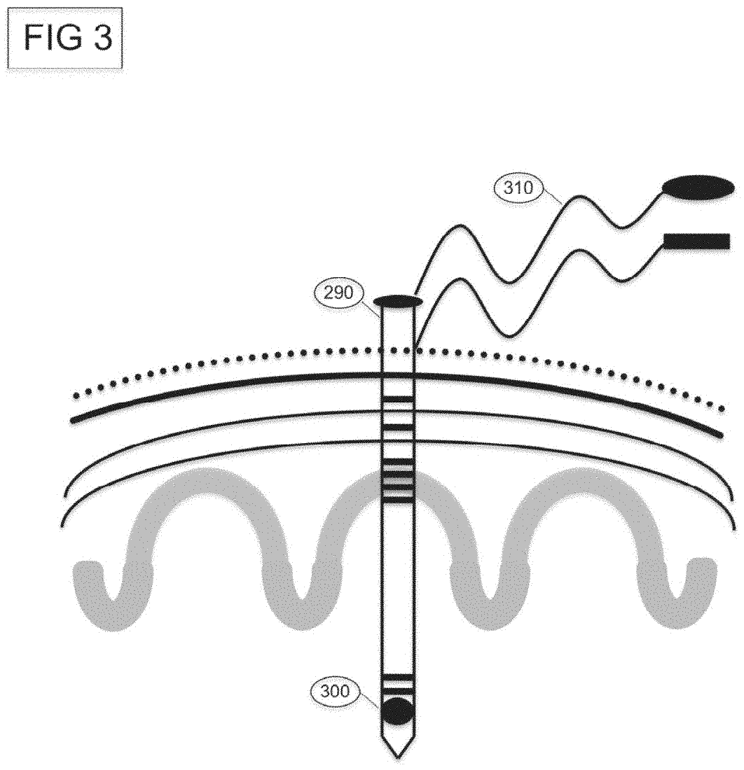

A further embodiment is shown in FIG. 3. In this case, the implanted device (290) comprises both recording elements (100) as described in FIG. 1 and a physiological sensor (300). In this example, the physiological sensor (300) on the implanted device (290) measures the intracranial pressure. In preferred embodiments, the implanted device (290) comprises recording elements (100) positioned to identify the Epidural space (40), the Subdural space (50), the Subarachnoid space (60), the Gray Matter of Cerebral Cortex (70), and the White Matter (80) compartments as shown in FIG. 1. Preferably, the recording elements (100) positioned in proximity to the tip of the implanted device (290) are co-located with the physiological sensor (300) to allow confirmation of position within the brain.

As exemplified in in FIG. 3, brain activity received from the recording elements (100) can be transferred to the interface (150) as described, for example in FIG. 2. Additionally, in one embodiment, the data received from the physiological sensor (300) can be transferred to a separate hardware element (310) capable of processing the physiological parameter data, and in this case, data relating to intercranial pressure.

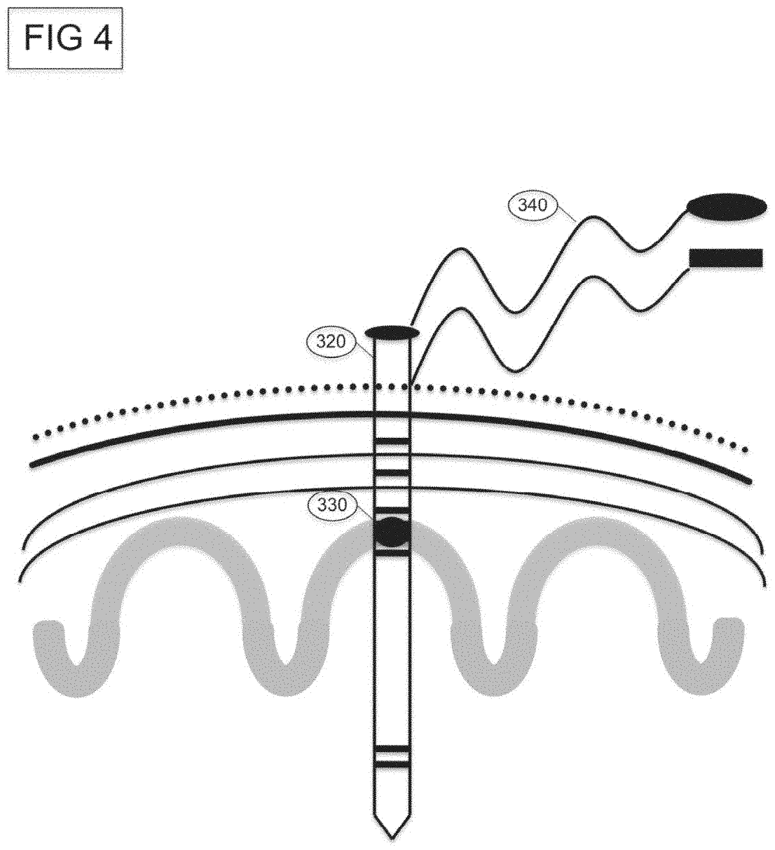

A further embodiment is shown in FIG. 4. In this case, the implanted device (320) comprises both recording elements (100) as described in FIG. 1 and a physiological sensor (330) positioned at a different location on the shaft of the implanted device (320) as compared to FIG. 3. In this example, the physiological sensor (330) on the implanted device (320) measures the temperature in the Grey Matter of the Cerebral Cortex (70). In preferred embodiments, the implanted device (320) comprises recording elements (100) positioned to identify the Epidural space (40), the Subdural space (50), the Subarachnoid space (60), the Gray Matter of Cerebral Cortex (70), and the White Matter (80) compartments as shown in FIG. 1. Preferably, the recording elements (100) positioned on the implanted device (320) are co-located with the physiological sensor (330) to allow confirmation of position within the brain.

As exemplified in in FIG. 4, brain activity received from the recording elements (100) can be transferred to the interface (150) as described, for example in FIG. 2. Additionally, in one embodiment, the data received from the physiological sensor (330) can be transferred to a separate hardware element (340) capable of processing the physiological parameter data, and in this example, data relating to temperature of the Grey matter.

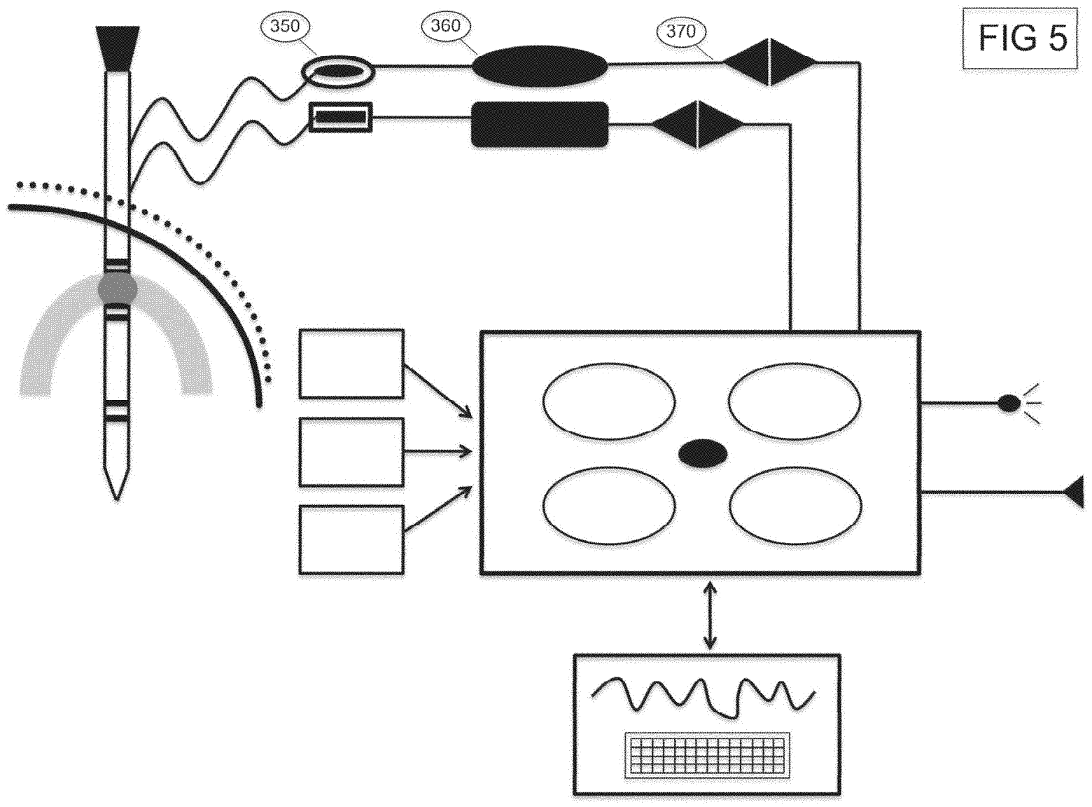

FIG. 5 exemplifies how data obtained from the implanted device shown in FIG. 4 can be processed. In this example, brain activity data derived from the recording elements is transferred to the interface (150) as described in FIG. 2. In parallel, data derived from the physiological sensor (330) of FIG. 4 is transferred to the hardware element (170) comprising the processor (180) as shown in FIG. 2. In one embodiment, a means for transferring (350) the temperature sensor data to a temperature specific interface device (360) which is capable of processing the temperature sensor data recorded by the physiological sensor (330). A means for transferring (370) the processed temperature data to the hardware element (170) is also shown in FIG. 5.

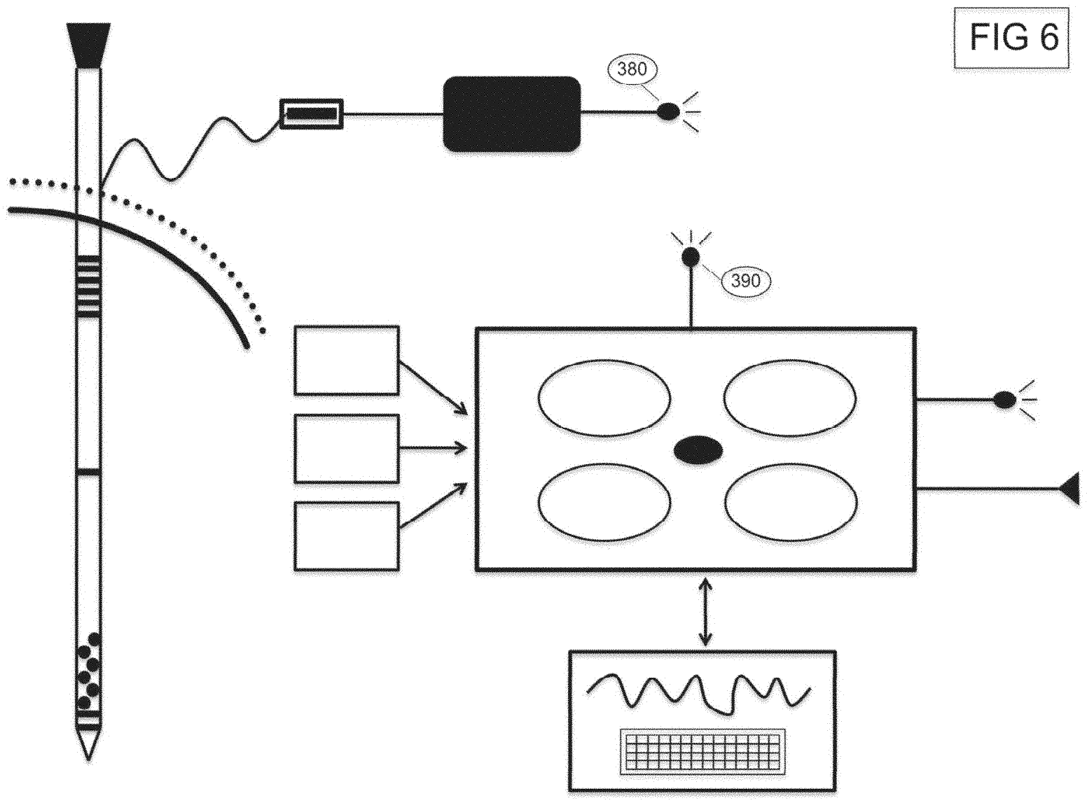

FIG. 6 exemplifies a preferred embodiment of transmitting the data obtained from the implanted device to the processor. In this embodiment, the brain activity data is transferred to the interface via a physical (e.g., wired) connection which is then transferred from the interface to the processor via a wireless transmitter (380) to a wireless receiver (390) on the hardware interface and/or processor.

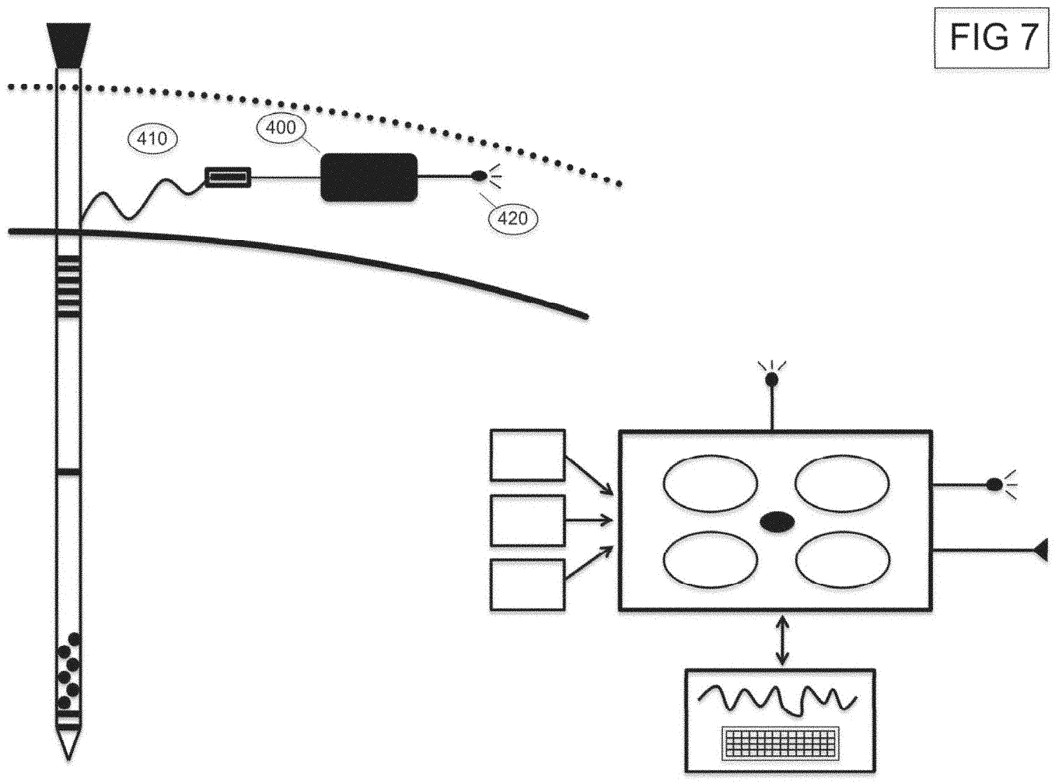

FIG. 7 exemplifies a further preferred embodiment wherein a modified interface (400) is capable of being implanted under the skin (410) of a patient. In this embodiment, the interface (400) comprises a wireless transmitter element (420) capable of communicating with a wireless receiver element associated with the hardware containing the processor, as exemplified in FIG. 6.

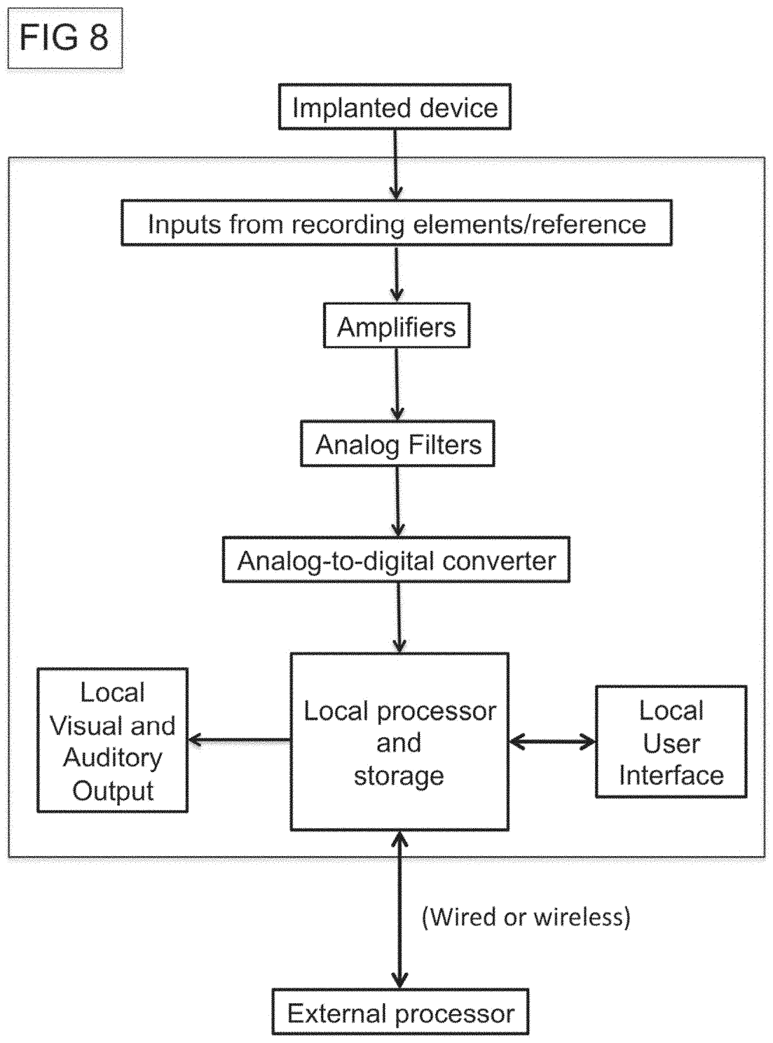

FIG. 8 is flow diagram outlining the steps associated with transmission and initial processing of recorded brain activity, detected by the recording elements on the implanted device, by the interface. In FIG. 8, the initial processing of brain activity is completed within the interface and then modified data is then transferred to the hardware element for final processing. However, it is envisioned that all processing of brain data can be accomplished by either the interface and/or the processor in isolation. As shown in FIG. 8, in one preferred embodiment of the interface, auditory and/or visual signals can be generated by the processor within the interface in response to particular patterns of brain activity.

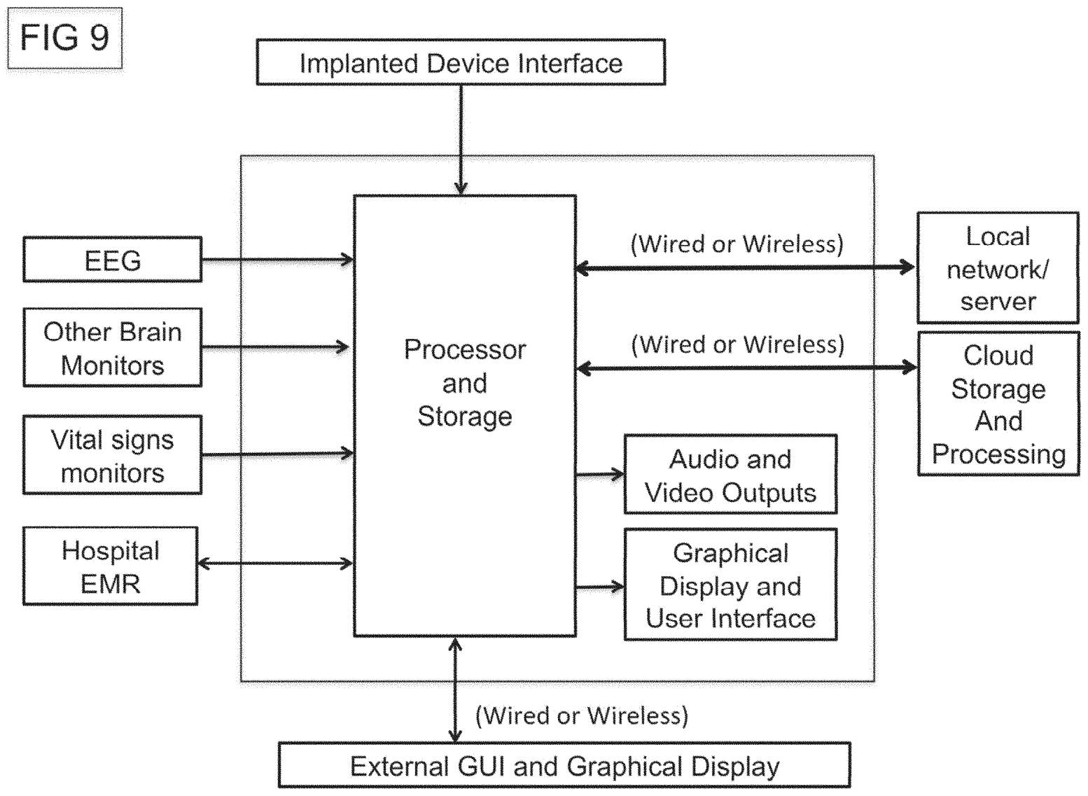

FIG. 9 is a flow diagram outlining potential components of the hardware element containing the processor, which includes the processor along with the various inputs and outputs for the described functions.

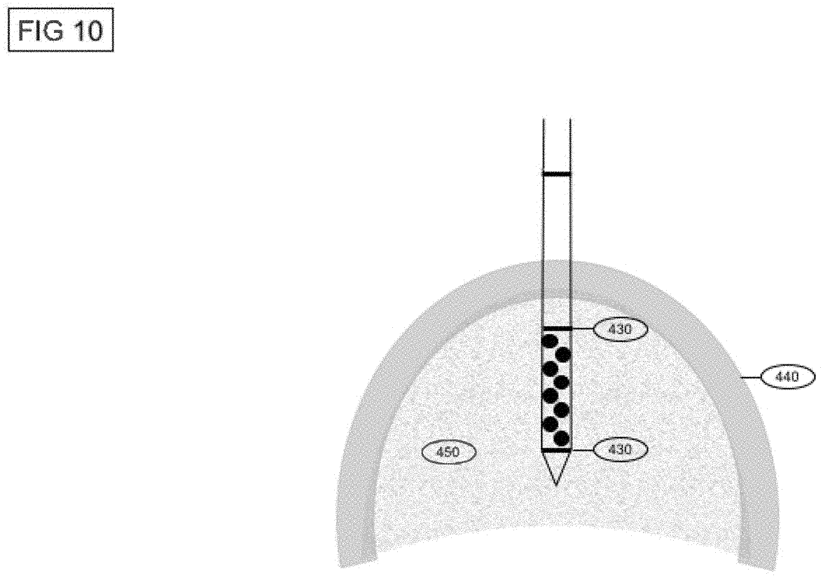

FIG. 10 exemplifies a further preferred embodiment wherein recording elements (430) located proximal and distal to a drainage function of a device designed to be placed through the ependyma (lining of the ventricle; 440) and completely within the CSF (450) of the cerebral ventricle are used to confirm similarity or dissimilarity for the purposes of confirming that the entirety of the drainage function resides within the cerebral ventricle.

### EXAMPLES

The invention will now be further illustrated with reference to the following examples. It will be appreciated that what follows is by way of example only and that modifications to detail may be made while still falling within the scope of the invention.

### Example 1: Position Demonstration

Presented below is representative data accumulated from a series of tests performed in adult pigs under the auspices of institutional Animal Care and Use Committee protocols. Animals were anesthetized using propofol and fentanyl and then a bilateral frontoparietal craniectomy was fashioned. The dura was opened widely to allow direct visualization of the surface of the cerebral cortex. Electrode insertion was performed under direct vision in a perpendicular trajectory to the brain surface at the apex of gyral curvature to ensure penetration down the length of the subtending gyrus. Subcortical electrode positioning was confirmed using a diagnostic ultrasound system equipped with a convex imaging array to visualize the sagittal plane to a depth of 5 cm, allowing a field of view extending from the cerebral cortex to the brainstem. This imaging strategy allowed for clear differentiation between cerebral cortex, subcortical white matter, cerebral ventricles, basal ganglia/thalamus, brainstem and cerebellum.

For recording brain electrical activity, standard clinical depth electrode arrays were acquired from Ad-Tech Corporation (Racine, Wis.) and PMT Corporation (Chinhassen Minn.). Eight-contact arrays were utilized with either 2 mm contact width and 5 mm center-to-center contact spacing or 1 mm contact width and 2 mm center-to-center contact spacing.

Electrode insertion was initiated in the midposition (anterior to posterior) of the superior or middle frontal gyrus and trajectories were directed under ultrasound guidance in a medial and posterior course to sequentially traverse the underlying anatomic compartments of interest (cerebral cortex, white matter, ventricle, and periventricular gray matter structures in sequential order). Following passage of the electrodes into brain tissue the electrodes were secured for prolonged recording, with position confirmed over time under direct vision. Ground and reference electrodes were placed in the contralateral subcutaneous tissue.

EEG data was recorded using a commercially available EEG headbox (Mitsar Co. Ltd, St. Petersburg Russia) and a standard PC laptop running Mitsar EEG Studio software. Data was then exported and analyzed off-line using the Insight software package (Persyst, Solana Beach Calif.) along with in-house analytical software. EEG data was analyzed using both referential (based on the common recording reference) and bipolar (adjacent contact-to-contact) methods. Exemplary images of raw waveform data were generated by screen capture. Amplitude (uv/Hz or sqrt uv/Hz) at select representative time points for the noted electrode pairs was generated using Fast Fourier Transform (FFT) of EEG data calculated over eight second epochs using overlapping sliding two-second windows. Spectrograms depict amplitude over time calculated from the noted electrode pairs in the 0-20Hz range using the pseudocolor scale (ordered in color spectrum with black/blue lowest power and red/white highest power) as shown.

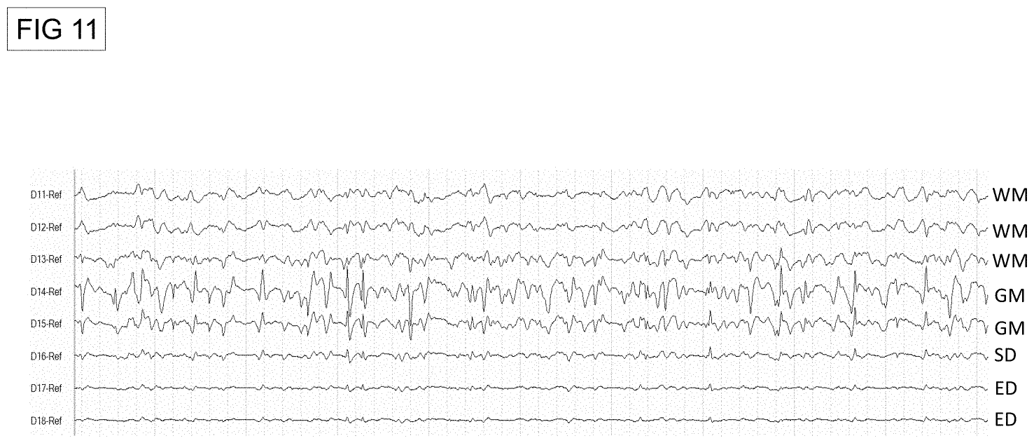

FIG. 11 demonstrates that anatomic compartments can be differentiated based on waveform analysis of EEG recorded from multicontact electrode arrays as described herein with an extracranial common reference electrode. FIG. 11 provides representative data for this approach, demonstrating that EEG waveforms with the largest signal amplitude and higher-frequency activity can be localized to the gray matter of the cerebral cortex (GM) (within which the EEG signal is biologically generated). Progressively smaller signals can be recorded from subcortical white matter (WM), which is related to spread of the signals from cortical generators located in the gray matter (GM), and in similar fashion to the subdural space (SD) and epidural space (ED). Taking into consideration the known architecture of the brain along with predicted spacing of the recording contacts along the electrode array, the anatomic positioning of the array can thus be determined.

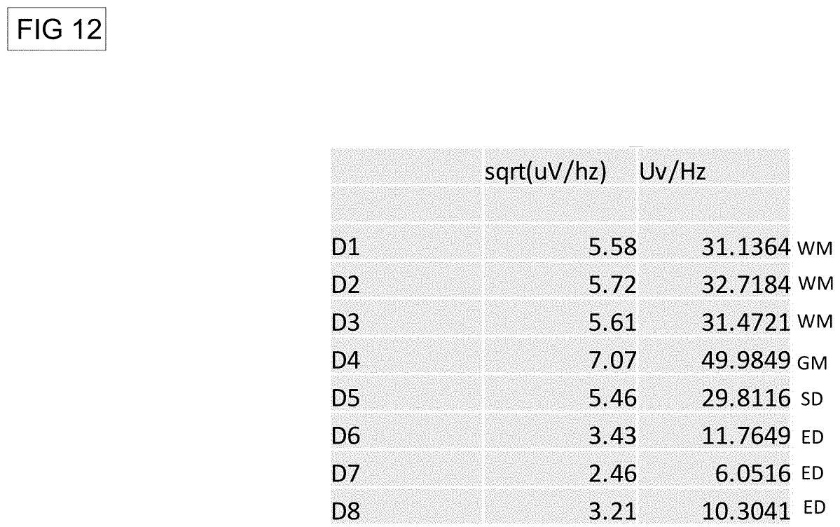

FIG. 12 demonstrates another preferred embodiment which utilizes quantitative comparison of potentials recorded from multicontact electrode arrays with an extracranial common reference to determine position of individual electrodes within specific intracranial compartments. As demonstrated in the chart within FIG. 12, the channel demonstrating the highest signal is located with the gray matter (GM) while sequentially smaller potentials are noted in the subcortical white matter (WM), subdural space (SD) and epidural space (ED). In this example, the gray matter potentials are noted to be 60.6%, 68.7%, and 385.2% larger than white matter, subdural space, and epidural space potentials respectively.

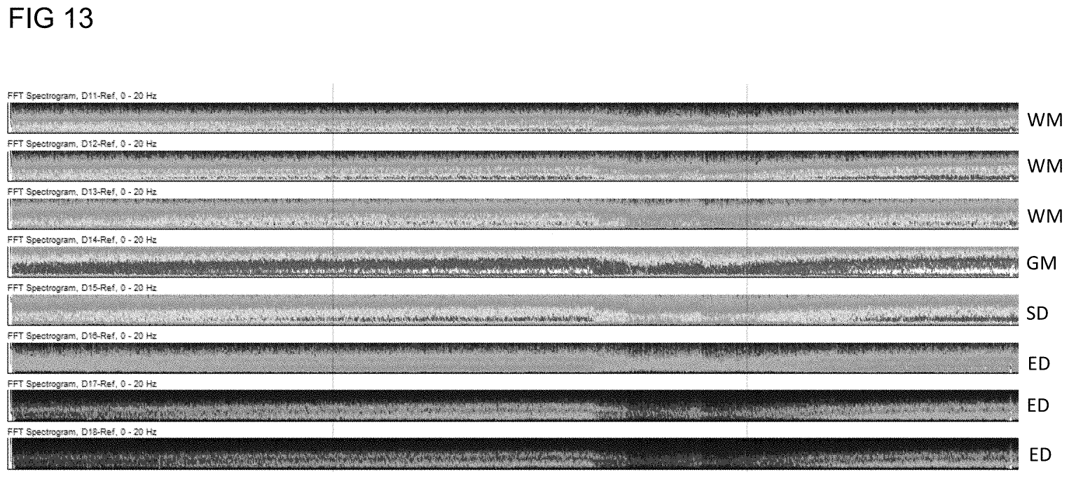

FIG. 13 demonstrates that electrodes within discrete intracranial compartments can be visually identified using compressed spectral analysis generated through Fast Fourier Transform of data from contacts along electrode arrays using a common extracranial reference. In a representative example displayed in FIG. 13, an electrode located in gray matter demonstrates significantly higher power (as evidenced by a preponderance of high-power “red” signal) than is seen in white matter, subdural space or epidural space (demonstrating a preponderance of lower power “blue” signal).

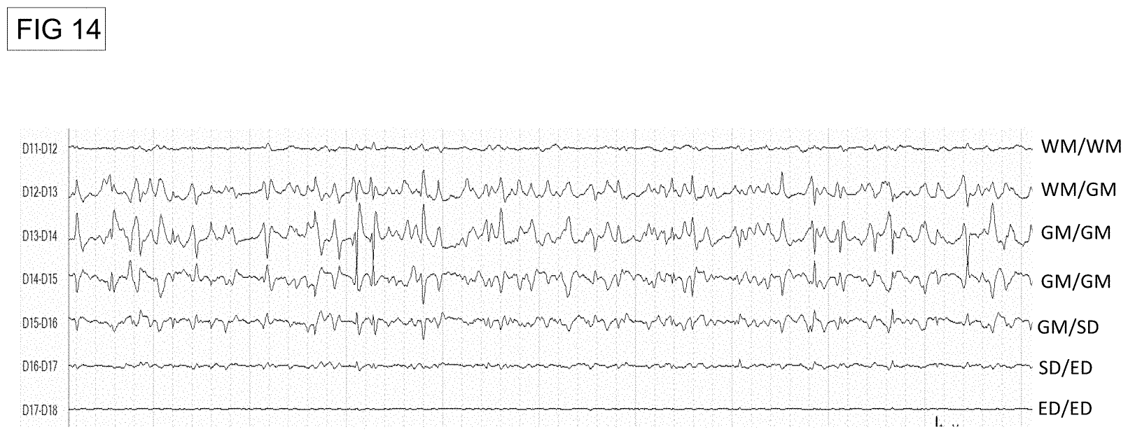

FIG. 14 demonstrates that bipolar referencing can alternatively be used to augment differences in electrical signals recorded from adjacent contacts that may lie within or near adjoining intracranial anatomic compartments. This strategy can reduce signal differences from electrodes located in bioelectrically identical regions while amplifying signals from electrodes spanning regions with higher bioelectrical diversity. This can be seen in raw EEG outlined in FIG. 14, where significantly smaller waveforms are observed when using bipolar referencing of adjacent contacts located in subcortical white matter (WM/WM) when compared to potentials seen at the white matter/gray matter junction (WM/GM) or within the gray matter itself (GM/GM). As EEG is demonstrates signals that can be bidirectionally recorded as a result of the dipole, recorded potentials from contacts bridging or including the “generator” demonstrate the highest signal amplitude.

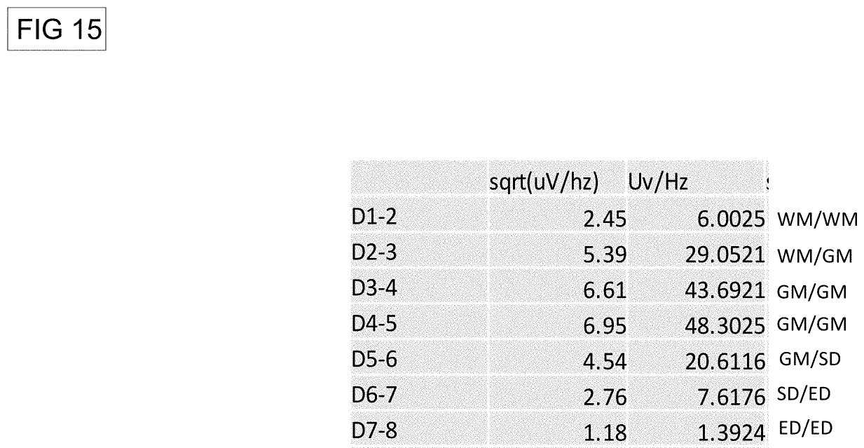

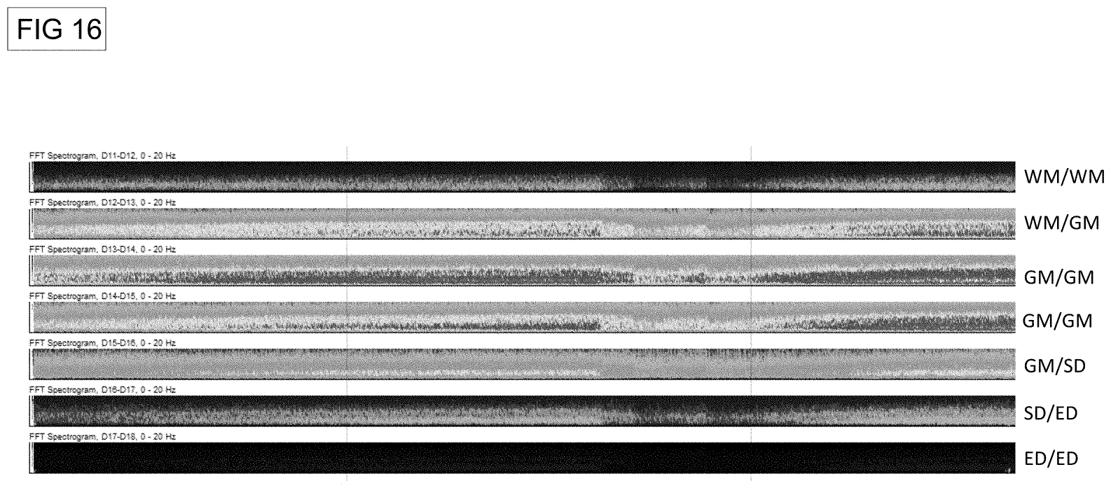

FIG. 15 demonstrates a second way that bipolar electrode referencing can be used to quantitatively analyze signals recorded from an electrode array. FIG. 15 demonstrates significant differences from electrode pairs located within discrete or adjoining intracranial compartments. The bipolar referencing strategy used to generate the data in FIG. 15 resulted in significantly higher electrical potentials recorded from GM/GM pairs when compared to WM/GM, GM/SD, WM/WM, SD/ED or ED/ED pairs. These differences can also be seen using compressed spectral array documenting quantitative analysis of total power in electrode pairs located within the anatomic compartments of interest, as exhibited in FIG. 16.

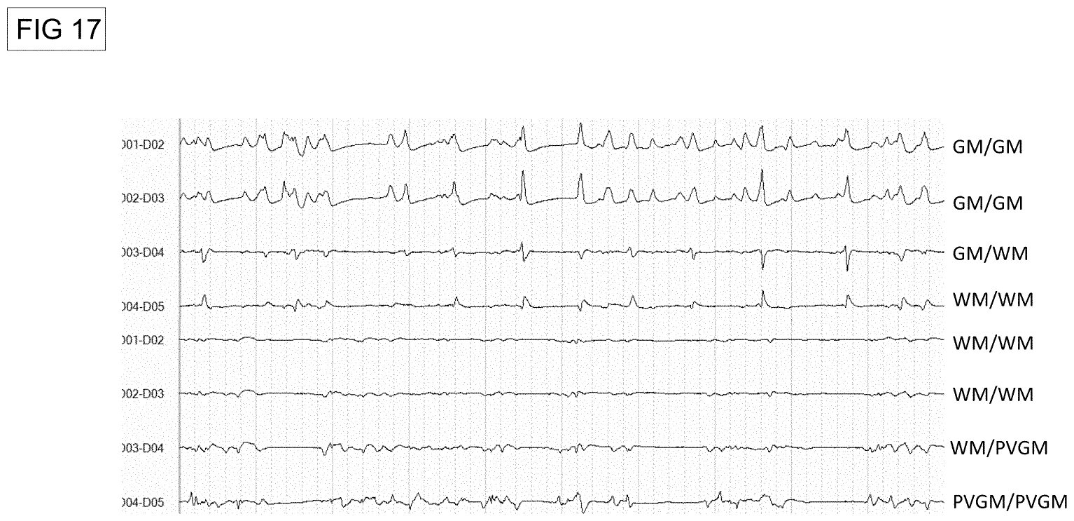

The data shown in FIG. 17 also demonstrates that a bipolar referencing strategy can be used to identify multiple intracranial compartments by taking advantage of known inter-contact electrode spacing along with consistencies of intracranial anatomy. This approach is exemplified in raw EEG data recorded from an electrode array spanning from the cerebral cortex to the periventricular gray matter, displayed in FIG. 17, in which bipolar channels extending from the cerebral cortex through the subcortical white matter to the periventricular gray matter demonstrate characteristic high-amplitude and higher-frequency signals in gray matter of cerebral cortex (GM), low-amplitude signals in subcortical white matter (WM), and higher amplitude but lower frequency signals in periventricular gray matter (PVGM).

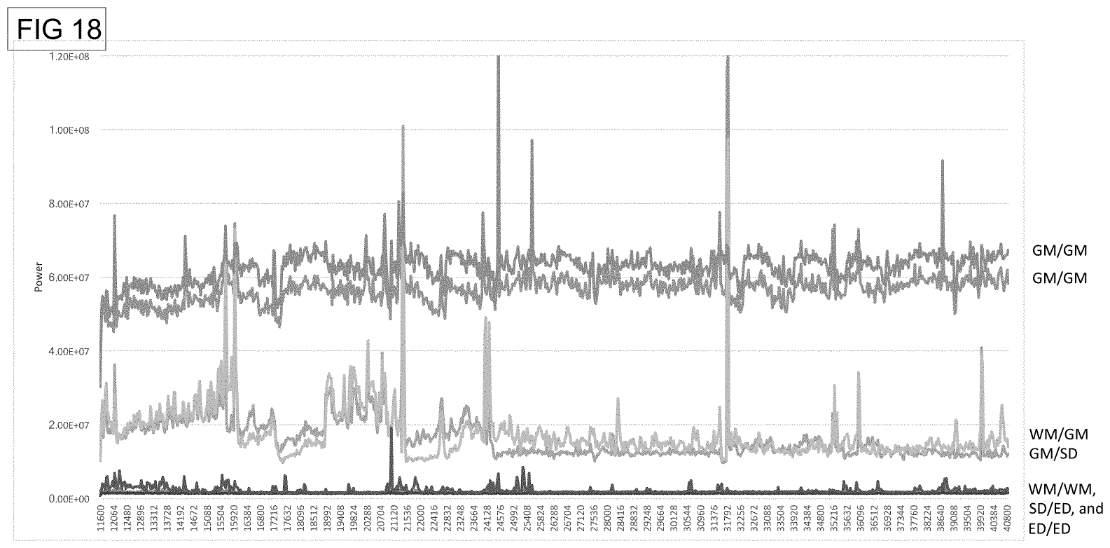

FIG. 18 demonstrates that stability of an electrode or electrode pair in an intracranial compartment over time can be performed using comparative analysis of total power recorded from each electrode pair. As exemplified in FIG. 18, an approach using bipolar referencing (adjacent contacts) of an electrode array spanning the cerebral cortex demonstrates high-power signals recorded in GM/GM contacts with lesser power signals recorded over time from WM/GM and GM/SD pairs. Significantly lower power is consistently seen in electrode pairs spanning WM/WM, SD/ED and ED/ED compartments.

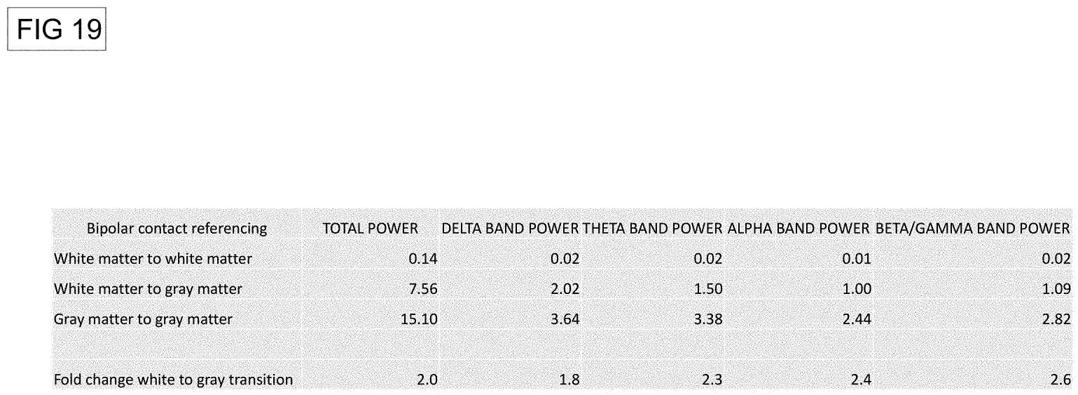

EEG power can be performed in specific frequency bands to augment the identification of compartments with highly divergent electrical activity. An example of this approach is provided in FIG. 19, in which analysis of total power between white and gray matter-associated contacts demonstrates a fold-change of 2.0, while this detected difference can be augmented by focusing on analysis of power within the beta/gamma band (13-30 Hz) which demonstrates a fold-change of 2.6.

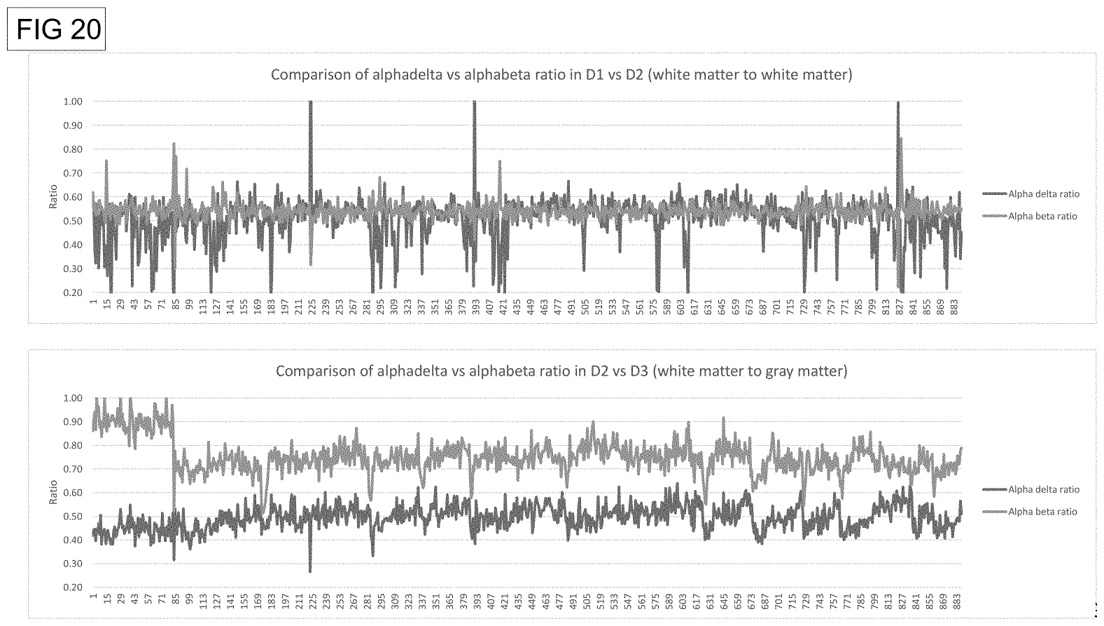

Additionally, comparative analysis of spectral ratios can identify electrode position within discrete intracranial compartments. As exhibited in FIG. 20, differential comparison of alpha/delta and alpha/betagamma ratios exposes highly parallel values in the white matter pairs while a significantly higher alpha/betagamma ratio than alpha/delta ratio is seen in the pair spanning the gray-white junction (due to the more focal presence of faster frequencies within the gray matter of the cerebral cortex).

1. A system for detecting the position of an implanted device within or around a compartment of a brain, wherein said system comprises an implanted device comprising a recording element capable of detecting and transmitting in real time brain activity connected by an interface to a processor capable of analyzing the position of the implanted device within or around the compartment of the brain.

2. The system of claim 1, wherein the compartment of the brain is selected from:
(a) gray matter;
(b) white matter;
(c) cerebral ventricle or other fluid containing space;
(d) transition zone between gray matter and white matter;
(e) transition zone between gray matter and a cerebral ventricle;
(f) transition zone between white matter and a cerebral ventricle;
(g) subdural or subarachnoid space;
(h) epidural space;
(i) local vasculature;
(k) transition between bone, epidural space, subdural space, subarachnoid space, brain tissue, or fluid-containing space;
(l) position within a specific geographic area of the brain in relationship to other structures or devices (including but not limited to anterior/posterior, medial/lateral, superior/inferior);
(m) triangulated position of devices using data recorded from multiple sources; or
(n) device proximity to or distance from any one of the compartments of (a)-(k).

3. The system of claim 1, wherein the brain activity is measured by: at least one parameter selected from:
(a) average voltage level;
(b) root mean square (rms) voltage level and/or a peak voltage level;
(c) derivatives involving fast Fourier transform (FFT) of recorded brain activity, including spectrogram, spectral edge, peak values, phase spectrogram, power, or power ratio; also including variations of calculated power such as average power level, rms power level and/or a peak power level;
(d) measures derived from spectral analysis such as power spectrum analysis; bispectrum analysis; density; coherence; signal correlation and convolution;
(e) measures derived from signal modeling such as linear predictive modeling or autogressive modeling;
(f) integrated amplitude;
(g) peak envelope or amplitude peak envelope;
(h) periodic evolution;
(i) suppression ratio;
(j) coherence and phase delays;
(k) wavelet transform of recorded electrical signals, including spectrogram, spectral edge, peak values, phase spectrogram, power, or power ratio of measured brain activity;
(l) wavelet atoms;
(m) bispectrum, autocorrelation, cross bispectrum or cross correlation analysis;
(n) data derived from a neural network, a recursive neural network or deep learning techniques; or
(0) identification of the recording element(s) detecting local minimum or maximum of parameters derived from (a-n);
preferably wherein said brain activity is measured by categorical measures of values selected from volts (V), hertz (Hz), and/or derivatives and/or ratios thereof, and preferably, wherein (1) said categorical measures indicate a change in brain activity of, for example at least 10%, of at least 20%, of at least 30%, of at least 40%, of at least 50%, of at least 90%, or at least 99% and represents transition from:
(a) gray matter to white matter;
(b) gray matter to cerebral ventricle;
(c) white matter to cerebral ventricle;
(d) subdural/subarachnoid space to gray matter/white matter
(e) epidural space to subdural/subarachnoid space or gray matter/white matter
(f) cerebral vasculature in one compartment to cerebral vasculature in another compartment;
(g) a compartment (either normal or pathological) within the brain other than gray matter, white matter, or cerebral ventricle such as an arachnoid cistern, intracerebral cyst, hematoma, tumor tissue, products of infection, or
(h) any combination of the above compartments,
or (2) wherein the categorical measures indicate a change in brain activity of, for example at least 10%, of at least 20%, of at least 30%, of at least 40%, of at least 50%, of at least 90%, or at least 99% and represents the implanted device being positioned outside of the gray matter.

4-6. (canceld)

7. The system of claim 1, wherein the implanted device:
(a) further comprises a physiological sensor, a preferably comprises more than one physiological sensor capable of measuring a physiological parameter, preferably wherein said physiological parameter is selected from intracranial pressure, oxygen concentration, glucose level, blood flow or tissue perfusion, tissue temperature, electrolyte concentration, tissue osmolarity, a parameter relevant to brain function and/or health, or any combination thereof;
(b) is designed for temporary, acute, semi-chronic, or chronic/permanent implantation in a patient;
(c) further comprises a therapeutic function, preferably wherein the therapeutic function is selected from:
(1) the ability to drain or access a biological fluid, such as CSF, cyst fluid, or hematoma (i.e., a drainage function);
(2) the ability to deliver a therapeutic agent;
(3) the ability to deliver an electrical signal;
(4) the ability to remove or ablate tissue; and/or
(5) any combination of (c)(1)-(c)(4);
(d) is constructed from plastic, metal, organic, inorganic, and/or alternate compounds appropriate for implantation into the body;
(e) incorporates and/or is impregnated with a therapeutic substance, preferably wherein said the therapeutic substance is an antibiotic;
(f) is flexible or rigid;
(g) is placed through the skin, bone, dura, brain tissue, fluid spaces, cerebral blood vessels or other body tissues;
(h) comprises more than one recording element;
(i) further comprises a reference sensor capable of measuring a reference parameter;
(j) comprises more than one reference sensor;
(k) comprises more than one physiological sensor;
(l) any combination of (a)-(k).

8-18. (canceled)

19. The system of claim 1, wherein the recording element is located:
(a) in proximity with the tip of the implanted device;
(b) in proximity with the structural portion of the implanted device designed to be positioned within the gray matter of the brain;
(c) in proximity with the structural portion of the implanted device designed to be positioned within the white matter of the brain;
(d) in proximity with the structural portion of the implanted device designed to be positioned within the subdural/subarachnoid space:
(e) in proximity with the structural portion of the implanted device designed to be positioned within the epidural space:
(f) in proximity with the structural portion of the implanted device designed to be positioned within a cerebral ventricle or other fluid containing space;
(g) in proximity with the structural portion of the implanted device designed to be positioned within a blood vessel;
(h) in proximity to the drainage function; and/or
(i) any combination of the above.

20. (canceled)

21. The system of claim 7, wherein the implanted device comprises more than one recording element, and wherein the more than one recording elements are positioned in patterns selected from:
(a) dispersed along the implanted device;
(b) located at the tip of the implanted device;
(c) located with a first recording element at least 50 um, 100 um, 200 um, 500 um, 750 um, 1 mm, 2 mm, 3 mm, 4 mm, 5 mm, 6 mm, 7 mm, 8 mm, 9 mm, 10 mm, 2 cm, 3 cm, 4 cm, 5 cm, 6 cm, 7 cm, 8 cm, 9 cm, 10 cm or any intervening distance apart from another recording element;
(d) located with a first recording element located in the white matter and a second recording element located in the gray matter;
(e) located with a first recording element located in the white matter, a second recording element is located in the grey matter, and a third recording element is located in a cerebral ventricle or other fluid space;
(f) physically separated from the implanted device and located elsewhere in/on the body or brain; and/or (g) located proximal and/or distal to the drainage function.

22-23. (canceled)

24. The system of claim 7, wherein the implanted device comprises more than one reference sensor, and wherein the more than one reference sensors are:
(a) dispersed along the implanted device;
(b) located at the tip of the implanted device;
(c) located with a first reference sensor at least 50 um, 100 um, 200 um, 500 um, 750 um, 1 mm, 2 mm, 3 mm, 4 mm, 5 mm, 6 mm, 7 mm, 8 mm, 9 mm, 10 mm, 2 cm, 3 cm, 4 cm, 5 cm, 6 cm, 7 cm, 8 cm, 9 cm, 10 cm or any intervening distance apart from a second reference sensor;
(d) located with a first reference sensor in the white matter and a second reference sensor located in the gray matter;
(e) located in a cerebral ventricle or other fluid containing space; and/or
(f) physically separated from the implanted device and located elsewhere in/on the body or brain.

25. (canceled)

26. The system of claim 7, wherein the implanted device comprises more than one physiological sensor, and wherein the more than one physiological sensors are:
(a) dispersed along the implanted device;
(b) located at the tip of the implanted device;
(c) located with a first physiological sensor at least 50 um, 100 um, 200 um, 500 um, 750 um, 1 mm, 2 mm, 3 mm, 4 mm, 5 mm, 6 mm, 7 mm, 8 mm, 9 mm, 10 mm, 2 cm, 3 cm, 4 cm, 5 cm, 6 cm, 7 cm, 8 cm, 9 cm, 10 cm or any intervening distance apart from a second physiological sensor;
(d) located with a first physiological sensor located in the white matter and a second physiological sensor is-located in the gray matter;
(e) located in a cerebral ventricle or other fluid containing space; and/or
(f) physically separated from the implanted device and located elsewhere in/on the body or brain.

27. (canceled)

28. The system of claim 1, wherein:
(a) the processor is capable of processing, filtering, amplifying, digitally transforming, comparing, storing, compressing, displaying, and/or otherwise transmitting (1) the brain activity; (2) the brain activity and the physiological parameter; (3) the brain activity, the physiological parameter, and the reference parameter; or (4) the brain activity and the reference parameter;
(b) the system updates in continuous or real-time fashion;
(c) the system concurrently detects and processes: (1) brain activity in more than one brain compartment; or (2) brain activity and a physiological parameter in more than one brain compartment;
(d) the processing of (1) brain activity or (2) brain activity and a physiological parameter occurs simultaneously;
(e) the implanted device, the interface, and the processor are integrated with one another; or the processor and the interface are integrated with one another; or the implanted device and the interface are integrated with one another;
(f) the interface is a physical interface; or the interface is a wireless interface;
(g) the interface is implanted within the patient; and/or
(h) interface is capable of processing, filtering, amplifying, digitally transforming, compressing and/or transmitting: (1) the brain activity; (2) the brain activity and the physiological parameter; (3) the brain activity, the physiological parameter, and the reference parameter; or (4) the brain activity and the reference parameter (i) or any combination of (a)-(h).

29-33. (canceled)

34. The system of claim 1, wherein the system measures:
(1) brain activity by: (a) recording brain activity from an optimal recording element; (b) minimizing and/or disregarding brain activity recorded from a sub-optimal recording element; or (c) a combination of (a) and (b); and/or
(2) a physiological parameter by: (a) recording the physiological parameter from an optimal physiological sensor; (b) minimizing and/or disregarding the physiological parameter recorded from a sub-optimal physiological sensor; or (c) a combination of (a) and (b);
and wherein preferably the measure of (a) brain activity or (b) brain activity and the physiological parameter occurs in real time and/or is dynamically adjusted to continuously identify optimal vs. sub-optimal elements or sensors.

35-36. (canceled)

37. The system of claim 1, wherein the system further comprises:
(a) an independent power source;
(b) at least one alternate physiological monitoring device, preferably wherein the alternate physiological monitoring device is connected to the system via a second interface; preferably wherein the second interface is either a physical or a wireless interface;
(c) a display component;
(d) means for providing auditory or visual information;
(e) a configuration for wireless transmission of data to a local server or cloud-based system; and/or
(f) a graphical user interface (GUI).

38. The system of claim 1-37, wherein the processor:
(a) further comprises hardware and/or software that analyzes, manipulates, displays, correlates, stores and/or otherwise transmits (a) the brain activity; (b) the brain activity and the physiological parameter; (c) the brain activity, the physiological parameter, and the reference parameter; or (d) the brain activity and the reference parameter; and/or
(b) is capable of inputting, recording, integrating, analyzing, compressing, storing, displaying, transmitting and/or utilizing data selected from:
(1) alternate physiological monitoring device implanted within or around the brain, including, but not limited to an oxygen sensor, a blood perfusion sensor, a brain metabolites sensor, a temperature sensor, or an intracranial pressure sensor;
(2) a system designed for monitoring aspects of physiology not directly recorded from the brain, including, but not limited to: heart rate monitor; EKG measurement device; temperature sensor; combined heart rate and oximeter device such as a pulse oximeter; blood pressure measurement device; or other physiological recording system and combinations thereof;
(3) a source of electroencephalography or electrocorticography such as that recorded from standard scalp or subdural electrodes;
(4) associated clinical interventions such as medications, ventilator settings, or temperature management; or
(5) a patient's medical record.

39. The system of claim 38, wherein the hardware further comprises a power supply, a central processing unit/motherboard, memory components, data/media storage capacity, video/graphics card, a sound card, input and output peripherals, physical connections for wired transmission and/or a wireless interface.

40-42. (canceled)

43. The system of claim 37, wherein the system comprises at least one alternative physiological monitoring device, the alternative physiological monitoring device is selected from: a heart rate monitor, a EKG measurement device, an oximeter, combined heart rate and oximeter device such as a pulse oximeter, a body temperature sensor, a blood pressure measurement device, a neuronal activity measurement device, an EEG measurement device, or other physiological recording systems and combinations thereof.

44-45. (canceled)

46. The system of claim 37, wherein the system further comprises a display component and wherein the display component is capable of displaying:
(a) at least one native or processed brain activity detected by the implanted device;
(b) at least one aspect of brain physiology detected by a concurrent physiological monitor associated with the implanted device;
(c) at least one aspect of brain physiology detected by a physiological monitor directly associated with the brain not directly associated with the system;
(d) at least one aspect of other physiological data recorded from the patient not directly associated with the brain, such as heart rate, systemic oxygen saturation, blood pressure or other vital signs;
(e) at least one aspect of other clinical information associated with the patient, such as demographic data or medications being administered;
(f) data associated with the function of the system as a whole, such as specific recording elements in use, location of selected recording elements within particular compartments of the brain, specifics regarding analytics of displayed brain activity, system power level and/or related variables;
(g) the brain activity;
(h) the brain activity and the physiological parameter;
(i) the brain activity, the physiological parameter, and the reference parameter; and/or
(j) the brain activity and the reference parameter.

47. (canceled)

48. The system of claim 37, wherein the system comprises a means for providing auditory or visual information, wherein the auditory or visual information provides information selected from:
(a) the position of the implanted device or an aspect of the implanted device within a compartment of the brain;
(b) the settings or function of the system;
(c) changes associated with monitored brain activity or associated physiological variables;
(d) factors controlled by the user with regard to the function or display capabilities of the system;
(e) visual information regarding implanted device position;
(f) auditory feedback regarding implanted device position;
(g) feedback enabling alteration of settings or performance of the system;
(h) the brain activity;
(i) the brain activity and the physiological parameter;
(j) the brain activity, the physiological parameter, and the reference parameter; or
(k) the brain activity and the reference parameter.

49. (canceled)

50. The system of claim 37, wherein the system further comprises a configuration of wireless transmission of data to a local server or cloud based system and wherein data is selected from:
(a) unprocessed or processed brain activity;
(b) other physiological monitors;
(c) documentation of associated clinical interventions;
(d) other patient-specific factors;
(e) the brain activity;
(f) the brain activity and the physiological parameter;
(g) the brain activity, the physiological parameter, and the reference parameter; and/or
(d) the brain activity and the reference parameter.

51. (canceled)

52. The system of claim 37, wherein the system comprises a graphical user interface (GUI), wherein the GUI allows the user to modify variables associated with the system, preferably wherein the variables are selected from:
(a) aspects of real-time feedback regarding the position of the implanted device;
(b) the ability to allow the user to select or modify elements of the display function;
(c) the ability to allow the user to select or modify elements of the recording or a reference function;
(d) the ability to allow the user to select or modify elements of the system processor with regard to aspects of recorded brain activity analysis;
(e) the ability to allow the user to enter additional data or patient information;
(f) the ability to allow the user to select or modify alarms or indicators; and/or
(g) the ability to allow the user to otherwise modify the input, output, storage, analytical, display or recording function of the system.

53. (canceled)

54. The system of claim 1, wherein the system further comprises software selected from:
(a) software designed to detect and display specific electrical patterns or signals of measured brain activity;
(b) software designed to calculate and display integrated amplitude of recorded electrical signals of measured brain activity;
(c) software designed to calculate and display peak envelope or amplitude peak envelope of recorded electrical signals of measured brain activity;
(d) software designed to calculate and display periodic evolution within recorded electrical signals of measured brain activity;
(e) software designed to calculate and display suppression ratios within recorded electrical signals of measured brain activity;
(f) software designed to calculate and display coherence and phase delays of measured brain activity;
(g) software designed to calculate and display fast Fourier transform of recorded electrical signals, such as for example, FFT; possibly including spectrogram, spectral edge, peak values, phase spectrogram, power, or power ratio of measured brain activity;
(h) software designed to calculate and display wavelet transform of recorded electrical signals, possibly including spectrogram, spectral edge, peak values, phase spectrogram, power, or power ratio of measured brain activity;
(i) software designed to calculate and display wavelet atoms associated with recorded electrical signals of measured brain activity;
(j) software designed to calculate and display bispectrum, autocorrelation, cross bispectrum or cross correlation analysis of recorded electrical signals of measured brain activity;
(k) software designed to calculate and display signals from isolated frequency bands of oscillatory electrical activity of measured brain activity;
(l) software designed to calculate and display ratios comparing elements of variation in particular frequency bands of oscillatory electrical activity of measured brain activity;
(m) software designed to calculate and display relative levels of activity in individual frequency bands of oscillatory electrical activity of measured brain activity;
(n) software that utilizes a neural network, a recursive neural network or deep learning techniques;
(o) software to identify a sensor recording local minimum or maximum of parameters derived from (a-n), for example as identified by waveform phase reversal in a bipolar chain of sequential adjacent sensors;
(p) software designed to record and/or measure the brain activity;
(q) software designed to record and/or measure the brain activity and the physiological parameter;
(r) software designed to record and/or measure the brain activity, the physiological parameter, and the reference parameter;
(s) software designed to record and/or measure the brain activity and the reference parameter; and/or
(t) software to measure changes in real time any one of the parameters derived from (a)-(s).
